(19) United States
(12) Patent Application Publication
(10) Pub. No.: US 2007/0082865 A1
(43) Pub. Date: Apr. 12, 2007

(54) Compositions and methods for treatment of airway hypersecretion

(75) Inventor: Michael J. Holtzman, Clayton, MO (US)

Correspondence Address: SENNIGER POWERS, ONE METROPOLITAN SQUARE, 16TH FLOOR, ST LOUIS, MO, 63102, US

(73) Assignee: Washington University, St. Louis, MO (US)

(21) Appl. No.: 11/545,701

(22) Filed: Oct. 10, 2006

Related U.S. Application Data
(60) Provisional application No. 60/725,396, filed on Oct. 11, 2005.

(51) Int. Cl. A61K 48/00; A61K 39/395

(52) U.S. Cl. 514/44; 424/145.1

(57) ABSTRACT

The invention relates generally to the field of treating pulmonary diseases. More specifically, the invention relates to the treatment of airway hypersecretion by the administration of an inhibitor of the epidermal growth factor receptor (EGFR) signaling pathway in combination with an inhibitor of the interleukin-13 (IL-13) signaling pathway, as well as compositions thereof.

### CROSS REFERENCE TO RELATED APPLICATIONS

This application claims the benefit of U.S. Provisional Patent Application Ser. No. 60/725,396, filed Oct. 11, 2005, the entire content of which is hereby incorporated herein by reference.

This invention was made with Government support under PO1 HL29594 awarded by NHLBI. The Government has certain rights in the invention.

### FIELD OF THE INVENTION

The present invention generally relates to compositions and methods for the treatment of airway diseases.

### BACKGROUND

Airway hypersecretion is a feature of airway diseases, including chronic obstructive pulmonary disease (COPD), cystic fibrosis, and asthma. In an individual suffering from hypersecretion, mucus accumulates in the airways and may cause airway obstruction. Airway submucosal glands and goblet cells lining the airway epithelium secrete mucus, an adhesive, viscoelastic gel composed of water, carbohydrates, proteins, and lipids. In a healthy individual, mucus is a primary defense against inhaled foreign particles and infectious agents. Mucus traps these particles and agents and facilitates their clearance while also preventing tissues from drying out. Small airways that contain many goblet cells as well as peripheral airways and which cannot be cleared by cough are particularly vulnerable to mucus accumulation and gradual obstruction by mucus. A substantial number of individuals suffer from airway hypersecretion, as it is associated with a number of airway diseases, including COPD, cystic fibrosis, and asthma as well as respiratory infections, including viral bronchitis and bronchiolitis. In the United States, approximately 14.2 million people have been diagnosed with COPD. Cystic fibrosis affects over 30,000 Americans and asthma affects 17 million Americans.

Conventional treatments for individuals suffering from airway hypersecretion include use of systemic or inhaled corticosteroids, anticholinergics, antibiotic therapy, bronchodilators (e.g., methylxanthines), sympathomimetics with strong β2 adrenergic stimulating properties, aerosol delivery of “mucolytic” agents (e.g., water, hypertonic saline solution), and oral administration of expectorants (e.g., guaifenesin). With regard specifically to cystic fibrosis, a more recent approach has been administering DNAse to reduce the viscosity of the DNA-rich mucus or sputum, such that the mucus is easier to clear from the airways (Shak, et al., Proc. Natl. Acad., 87:9188-9192, 1990; Hubbard, et al., N. Engl. J. Med., 326:812, 1991). Apart from medication, chest physical therapy consisting of percussion, vibration, and drainage are also used to clear mucus from the airways. As a last resort, lung transplantation may be an option for those with severe pulmonary disease. Many of the above described medications have serious side effects. For example, inhaled corticosteroids can cause thrush (a yeast infection of the mouth), cough, or hoarseness and systemic corticosteroids have even more severe side effects, such as delayed sexual development, changes in menstrual cycle, weight gain, and increased blood sugar (diabetes). The side effects of methylxanthines include severe nausea, tremors, muscle twitching, seizures, and irregular heartbeat.

The scheme for virus-inducible EGFR- and IL-13-dependent pathways controlling epithelial host and remodeling is shown, for example, in FIG. 28. EGFR activation with receptor dimerization and receptor tyrosine kinase phosphorylation leads to activation of three pathways: (1) Ra1 recruitment followed by c-Src activation that leads to Stat1 and Stat3 activation; (2) Shc/Grb2 recruitment followed by Sos, Ras, and c-Raf activation that lead to MEK1/2 activation of ERK1/2; and (3) Gab1 recruitment followed by PI3K activation that leads to generation of phosphatidylinositol-3,4,5-phosphate (PI-3,4,5-P3), activation of PDK1/2 and then Akt that inactivate proapoptotic factors (e.g., Bad). IL-13 signaling is also capable of activating ERK1/2 and PI3K as well as Stat6 that each contribute to upregulation of genes (CLCA and MUC) that promote cilia to goblet cell transdifferentiation. IL-13 signaling activates IRS1/2-dependent cascade to ERK1/2 and Stat6 that each contribute to upregulation of genes (CLCA and MUC) that promote cilia to goblet cell transdifferentiation. Under physiologic conditions, these pathways may (in conjunction with IFN-dependent activation of Stat1) lead to protection from viral infection, but if there is persistent activation in a susceptible genetic background, the same pathways may lead to ciliated cell hyperplasia and goblet cell metaplasia. Rationale use of specific inhibitors, e.g., EGFR and IL-13 receptor blockers, may fully restore normal epithelial architecture.

Activation of the epidermal growth factor receptor (EGFR) system by its ligands has been shown to lead to the synthesis of mucin in airway epithelial cells as well as to goblet cell metaplasia in rats (Takeyama, et al., Proc. Natl. Acad. Sci., 96:3081-3086, 1999). Furthermore, it has been shown that EGFR expression is sparse in the airways of healthy individuals, but the receptor is expressed in asthmatic individuals (Burgel and Nadel, Thorax, 59:992-996, 2004). A factor or pathway that stimulates expression of EGFR in the airway epithelium is the tumor necrosis factor-α (TNFα) pathway (Takeyama, et al., Proc. Natl. Acad. Sci., 96:3081-3086, 1999). Nadel et al., U.S. Pat. No. 6,270,747, disclose treating hypersecretion by administering an EGFR antagonist.

The interleukin-13 (IL-13) signaling pathway is associated with airway remodeling and hypersecretion. A decoy receptor for IL-13 (slL-13Rα2-Fc) was found to inhibit allergen-induced goblet cell formation in mice (Wills-Karp, et al., Science, 282:2258-2261). IL-13 has been shown to directly drive mucin gene expression in airway epithelial cells cultured under physiological conditions and in vivo (Laoukili, et al., J. Clin. Invest., 108:1817-1824, 2001; Kondo, et al., Am. J. Respir. Cell Mol. Biol., 27:536-541, 2002). It has also been reported that IL-13 stimulates the release of TGFα, which binds to and activates EGFR, from the membranes of human respiratory epithelial cells, (Booth, et al., Am. J. Respir. Cell Mol. Biol., 25: 739-743, 2001). Intratracheal instillation of IL-13 into the lungs of rats causes goblet cell metaplasia and increases mucin production via a complex mechanism according to which IL-13 induces the production of IL-8, leading to neutrophil recruitment (Shim, et al., Am. J. Physiol. Lung Cell Mol. Physiol., 280: L134-L140, 2001).

### SUMMARY OF THE INVENTION

Among the various aspects of the present invention is the provision of an improved treatment for airway hypersecretion. The pathogenesis for airway hypersecretion involves the EFGR and IL-13 signaling pathways, both of which play a role in goblet cell formation. Thus, by inhibiting both the EGFR and IL-13 signaling pathways, the airway epithelium can approach more fully its original architecture.

Briefly, therefore, the present invention is directed to a process of treating airway hypersecretion in an individual, the process comprising administering an inhibitor of the EGFR signaling pathway and an inhibitor of the IL-13 signaling pathway.

The invention is also directed to a composition for the treatment of airway hypersecretion comprising an inhibitor of the EGFR signaling pathway and an inhibitor of the IL-13 signaling pathway and a pharmaceutically acceptable carrier.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A, 1B, 1C, 1D, 1E, and 1F are representative photomicrographs of airway sections from C57BL/6J mice obtained at 21 days after inoculation with SeV or an equivalent amount of UV-inactivated SeV (SeV-UV) and then immunostained for EGFR and phospho-EGFR (p-EGFR) as well as competition by 50-fold antigen excess. Bar=20 μm. Methodology is further described in Example 1.

FIGS. 2A, 2B, 2C, 2D, 2E, 2F, 2G, 2H, and 2I are representative photomicrographs of airway sections obtained from mice at 21 days after inoculation with SeV and then subjected to immunofluorescent staining for EGFR, β-tubulin, CCSP, and MUC5AC alone and in combination. Primary anti-EGFR Ab binding was detected by anti-CY3 Ab (red fluorescence), while others were detected by anti-FITC Ab (green fluorescence). Bar=20 μm. Methodology is further described in Example 1.

FIGS. 3A, 3B, 3C, and 3D are representative photomicrographs of airway sections obtained at 21 days after inoculation with SeV or SeV-UV and then subjected to hematoxylin/eosin staining, immunofluorescent staining for β-tubulin-IV (green fluorescence) and CCSP (red fluorescence), and immunostaining for MUC5AC. Immunostaining with non-immune IgG gave no signal above background (data not shown). Bar=20 μm. Methodology is further described in Example 2.

FIGS. 4A and 4B are bar graphs showing corresponding quantitative data for conditions in FIG. 3 as well as SeV post inoculation Day 12 without treatment and SeV plus treatment with EKB-569 at Day 12 after inoculation for Days 10-21. Values represent mean+SEM, and a significant difference from SeV-UV control is indicated by (*). Methodology is further described in Example 2.

FIGS. 5A, 5B, and 5C are representative photomicrographs of airway sections obtained from C57BL/6J and Balb/cJ mice at indicated days after inoculation with SeV and then subjected to immunostaining for BrdU (FIG. 5A), phosphor-EGFR (FIG. 5B), and MUC5AC (FIG. 5C). Bar=20 μm. Methodology is further described in Examples 2 and 3.

FIG. 6 is a bar graph showing corresponding quantitative data for conditions in FIG. 5A. Values represent mean±SEM, and a significant difference from Day 0 is indicated by (*). Methodology is further described in Examples 2 and 3.

FIG. 7 is a bar graph showing corresponding quantitative morphometry for airway sections that were obtained from Balb/cJ mice at 21 days after inoculation with SeV or SeV-UV and then subjected to immunostaining for β-tubulin, CCSP, and MUC5AC. Values represent mean±SEM, and a significant difference from SeV-UV control is indicated by (*) Bar=20 μm. Methodology is further described in Examples 2 and 3.

FIGS. 8A, 8B, and 8C are representative photomicrographs of airway epithelial cell (mTEC) cultures placed under air-liquid interface conditions for 10 days followed by immunostaining for EGFR (top) or double immunofluorescence followed by confocal microscopy for β-tubulin and either EGFR or p-EGFR. Methodology is further described in Example 2.

FIG. 9 is an image of a Western blot analysis of mTEC cultures that were placed in basic medium for 1 day and then treated with EGF (1 or 10 ng/ml) for 10 min with or without concomitant inhibitor. Each inhibitor was added at maximal effective concentrations to the lower chamber for 6 h and the upper chamber for 2.5 h before addition of EGF to both chambers. For each condition, cell lysates with anti-EGFR, p-EGFR, phospho-Akt (p-Akt), or phospho-ERK1/2 (p-ERK1/2) Ab and detection by enhanced chemiluminescence. Methodology is further described in Example 2.

FIGS. 10A, 10B, 10C, and 10D are representative photomicrographs of mTEC cultures that were treated with vehicle or PD153035 (0.3 μM) for 7 days at 37° C. and then subjected to immunofluorescent staining for β-tubulin and Hoechst 33432. Bar=20 μm. Methodology is further described in Example 2.

FIG. 11 is a series of bar graphs showing quantitative analysis of β-tubulin staining cells (expressed as a % of total Hoechst-staining cells) with and without treatment with PD153035, LY294002, and PD98059 given at the indicated doses for 7 days. A significant difference is indicated by (*). Methodology is further described in Example 2.

FIGS. 12A, 12B, 12C, 12D, 12E, 12F, 12G, and 12H are representative photomicrographs of mTEC cultures that were treated with vehicle, PD153035 (0.3 μM), LY294002 (50 μM), and PD98059 (50 μM) for 3 days at 37° C. and then subjected to immunofluorescent staining for cleaved fragment of active caspase 3 (Act-C-3) or TUNEL reaction. Bar=20 μm. Methodology is further described in Example 3.

FIGS. 13A and 13B are bar graphs showing quantitative analysis of FIG. 12 for activate caspase-3 staining cells (expressed as % of total Hoechst staining cells) using treatment conditions from FIG. 12 as well as PD15305 plus zVAD-fmk (100 μM). Values represent mean+SEM, and a significant difference from vehicle alone is indicated by (*). Methodology is further described in Example 3.

FIG. 14 is an image of immunoblot analysis of activate caspase 3 (Act-C-3) and caspase 9 (Act-C-9) in cell lysates from mTEC cultures using treatment conditions from FIG. 12. Anti-caspase-9 antibody recognizes precursor (C-9) and the cleaved fragment of activate caspase-9 (Act-C-9). Methodology is further described in Example 3.

FIG. 15 is a bar graph showing flow cytometric analysis of JC-1 staining of mTEC cultures using treatment conditions from FIG. 12. Values represent % of cells with decreased mitochondrial membrane potential (Δψm) detected by shift from FL2 to FL1. Values represent mean±SEM, and a significant difference from vehicle alone is indicated by (*). Methodology is further described in Example 3.

FIG. 16 is representative photomicrographs of mTEC cultures treated with or without IL-13 (100 ng/ml for 5 days) and with or without subsequent PD153035 (0.3 μM for 3 days) and subjected to immunofluorescent staining for MUC5AC (red) and active caspase 3 (green), as well as counterstaining with Hoechst dye (blue). Methodology is further described in Example 4.

FIGS. 17A and 17B are bar graphs showing corresponding quantitative data for FIG. 16. Values represent mean±SEM for % of active caspase 3-positive goblet cells (MUC5AC⁺ active caspase 3⁺/MUC5AC⁺ cells) and non-goblet positive cells (total TUNEL staining cells/total Hoechst staining cells). A significant difference from vehicle control is indicated by (*) Methodology is further described in Example 4.

FIGS. 18A, 18B, 18C, and 18D are representative transmission electron micrographs for cultured mTECs before treatment (FIG. 18A) and then after treatment with IL-13 (100 ng/ml for 2 days at 37° C.) (FIGS. 18B-18D). Early cilia-goblet cells are identified with cilia that are visible on the surface of cells that also contain a few mucous granules (FIG. 18B); late cilia-goblet cells exhibit greater numbers of mucous granules in the cytoplasm (FIG. 18C); and mature goblet cells contain characteristic mucous granules with no cilia (FIG. 18D). Methodology is further described in Example 4.

FIGS. 19A, 19B, and 19C are representative photomicrographs of airway sections obtained from mice at 21 days after SeV inoculation and subjected to confocal immunofluorescence microscopy for β-tubulin (green) and MUC5AC (red). Arrows indicate ciliated cells staining for β-tubulin (c), goblet cells staining for MUC5AC (g), and cells staining for both β-tubulin and MUC5AC (cg). Methodology is further described in Example 4.

FIGS. 20A, 20B, and 20C are representative photomicrographs of airway sections obtained as in FIG. 19, but immunostained for p-EGFR (red) and MUC5AC (green). Arrows indicate ciliated cells staining for p-EGFR (c), goblet cells staining for MUC5AC (g), and cells staining for both p-EGFR and MUC5AC (cg). Methodology is further described in Example 4.

FIGS. 21A, 21B, and 21C are representative photomicrographs of airway secretions obtained FIG. 19, but immunostained for CCSP (green) and MUC5AC (red). Arrows indicate cells staining for CCSP (cc) or CCSP and MUC5AC (ccg). Methodology is further described in Example 4.

FIG. 22 is a bar graph showing quantitative analysis of MUC5AC expressing cells that also immunostained for CCSP or β-tubulin. Values represent mean±SEM, and a significant difference from corresponding SeV-UV control is indicated by (*). Methodology is further described in Example 4.

FIG. 23A, 23B, and 23C are bar graphs showing real-time PCR results for lung IL-13 (FIG. 23A), mCLCA3 (FIG. 23B), and MUC5AC (FIG. 23C) mRNA levels corrected for GAPDH control level at indicated times after SeV inoculation. Values represent mean±SEM, and a significant difference from corresponding SeV-UV control is indicated by (*). Methodology is further described in Example 4.

FIG. 24 is a bar graph showing quantitative analysis of β-tubulin (ciliated cells), CCSP (Clara cells), and MUC5AC (goblet cells) immunostaining in airways of mice infected with SeV and treated with slL-13 receptor antagonist (slL-13Ra2-Fc) or control IgG from on days 12, 14, 17, and 20 after inoculation. Bars=20 μm. A significant difference from corresponding IgG treatment is indicated by (*). Methodology is further described in Example 4.

FIGS. 25A, 25B, and 25C are representative photomicrographs from lung sections obtained from COPD patients and immunostained for β-tubulin, MUC5AC, or CCSP and viewed with immunofluorescence (FIG. 25A) or for β-tubulin and MUC5AC or CCSP and MUC5AC and viewed with laser confocal scanning microscopy (FIG. 25B and FIG. 25C, respectively). Arrows and outlines indicate goblet cells that express MUC5AC (g), Clara cells that express CCSP (cc), cilia-goblet cells that co-express β-tubulin and MUC5AC (cig), or goblet cells that co-express CCSP (ccg).

FIGS. 26A, 26B, and 26C are representative photomicrographs of human large airway epithelial cells (hLAECs) cultured from COPD patients, incubated with IL-13 (100 ng/ml) for 5 days, and then immunostained for γ-tubulin (red) (FIG. 26A), MUC5AC (green) (FIG. 26B), and both γ-tubulin and MUC5AC (FIG. 26C). Arrows indicate cells that immunostain for γ-tubulin and MUC5AC.

FIGS. 27A, 27B, 27C, and 27D are representative photomicrographs of hLAECs cultured from control (non-COPD) subjects, incubated with IL-13 for 1 day, immunostained as in FIG. 26, and then viewed with laser confocal scanning microscopy in x-y (FIG. 27A) and z-axis (FIGS. 27B-27D) views. Arrows indicate cells that immunostain for both γ-tubulin and MUC5AC.

FIG. 28 is a schematic demonstrating a scheme for virus-inducible EGFR- and IL-13-dependent pathways controlling epithelial host response and remodeling.

FIGS. 29A, 29B, 29C, and 29D are representative photomicrographs from endobronchial biopsy sections obtained from healthy control and asthmatic subjects. Sections were immunostained for EGFR and phospho-EGFR using the same methods as FIG. 1 and FIG. 2. Bar=20 μm. Methodology is further described in Example 2.

FIGS. 30A and 30B are images of Western blot analysis showing the effect of EKB-569 treatment in vitro (FIG. 30A) and in vivo (FIG. 30B). FIG. 30A is a Western blot image of cell lysates of human tracheobronchial epithelial cells (hTECs) cultured under air-liquid interface conditions and incubated with or without EGF (100 ng/ml) or with or without IL-13 (100 ng/ml) in the absence or presence of EKB-569 (1 μM for 10 min at 37° C.) and blotted with the indicated antibodies. FIG. 30B is a western blot image of lung lysates from C57BL/6J mice inoculated with SeV or SeV-UV and treated with or without EKB-569 on post inoculation Days 10-21. Blotting was with the indicated antibodies. Methodology is further described in Examples 1 and 2.

### DETAILED DESCRIPTION OF THE INVENTION

Immune signals, if left unchecked, can lead to an epithelial phenotype characteristic of chronic airway disease. In particular, persistent goblet cell metaplasia, leading to a chronic asthma/bronchitis disease phenotype, depends upon EGFR-dependent survival of ciliated epithelial cells and IL-13-dependent transdifferentiation of ciliated cells to goblet cells (see e.g. Examples 1 and 2). This abnormality can be corrected by targeted inhibition of signaling steps in the EGFR and IL-13 signaling pathways. Treatment with EGFR inhibitors allows for the ciliated cells to proceed toward programmed cell death while IL-13 blockade prevents the transition from ciliated cells to goblet cells (see e.g., Examples 2 and 3). Thus, as described herein, blocking of both the EGFR and IL-13 signaling pathway can restore the airway epithelium to its original architecture (see e.g., Example 4).

One aspect of the present invention, therefore, is the treatment, prophylactic or therapeutic, of epithelial hyperplasia and metaplasia through targeting the EGFR and IL-13 signaling pathways. Another aspect of the invention is compositions that target the EGFR and IL-13 signaling pathways, useful in the treatment of epithelial hyperplasia and metaplasia described herein. Such treatment can be used, for example, as a prophylactic to protect, in whole or in part, against chronic asthma and/or bronchitis. The compositions and treatments can also be used, for example, therapeutically to ameliorate altered epithelial architecture in the setting of asthma, bronchitis, bronchiolitis, and/or related inflammatory and infectious disorders characterized by a similar pattern of EGFR activation, IL-13 expression, and goblet cell metaplasia. The compositions and treatments can similarly be used to treat an airway disease or condition characterized by hypersecretion of mucus.

Disease states indicative of a need for therapy with inhibitors of EGFR and IL-13 signaling and disease states amenable to treatment with inhibitors of EGFR and IL-13 signaling include, for example, chronic obstructive pulmonary disease, nasal hypersecretory diseases (e.g., nasal allergies), inflammatory diseases (e.g., asthma, bronchiectasis, and pulmonary fibrosis), and chronic obstructive lung diseases (e.g., chronic bronchitis), as well as genetic diseases including cystic fibrosis, familial non-cystic fibrosis mucus inspissation of respiratory tract, Kartagener syndrome, alpha-1-antitrypsin deficiency, and upper or lower airway infections (e.g., viral bronchiolitis or rhinitis) that trigger or cause hypersecretory conditions.

A determination of the need for treatment will typically be assessed by a history and physical exam consistent with over production of mucus (e.g., cough productive of mucous), radiographic or other imaging studies of the airways that indicate diseases or conditions with overproduction of mucous, or pulmonary function tests that indicate evidence of airway obstruction and/or hyperreactivity.

In one aspect of the invention, the method comprises reducing the level of EGFR and IL-13 signaling in epithelial tissue by administering an inhibitor of EGFR signaling and an inhibitor of IL-13 signaling. The amount administered is at least that sufficient to prevent increases in ciliated cells and also prevent transdifferentiation of ciliated cells to goblet cells. In one exemplary study, treatment on days 12, 14, 17, and 20 post-viral infection with a decoy receptor that blocks IL-13 was effective in preventing virus-induced goblet cell metaplasia while daily treatment from post-infection days 10-21 with selective inhibitors of EGFR signaling caused a dose-dependent loss of ciliated cells out of proportion to the consequent decrease in total epithelial cells (see e.g. Example 4). These results demonstrate that treatment with inhibitors of EGFR and IL-13 signaling can correct epithelial architecture in the setting of inflammatory disease characterized by EGFR activation, IL-13 expression, and goblet cell metaplasia.

Signaling Inhibitors

Inhibitors of the EGFR or IL-13 signaling pathway can target, directly or indirectly, any factor or component involved in the biological cascade which results in promoting increases in ciliated cells and goblet cell metaplasia, respectively.

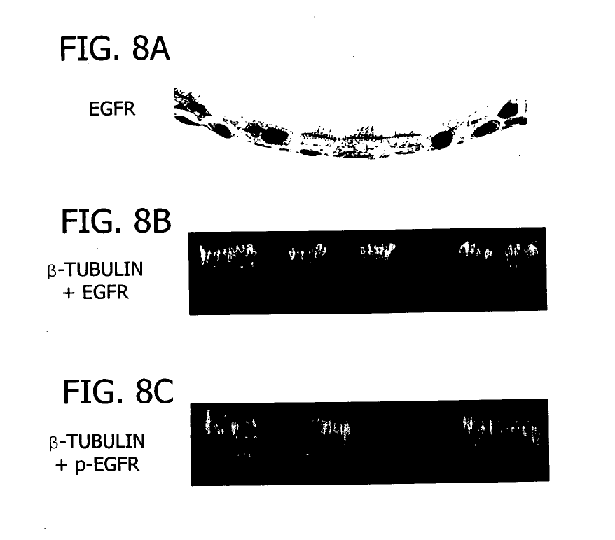

Inhibitors of EGFR signaling include inhibitors targeting EGF, EGFR, ligands of EGFR (e.g., amphiregulin, HB-BGF, and TGF-α), stimulators of EGFR expression, components of EGFR (e.g., hev-1 and hev-2), and signaling components downstream of EGFR. For example, an EGFR signaling inhibitor can target EGFR activation (e.g., receptor dimerization or receptor tyrosine kinase phosphorylation) or can target one or more of the pathways triggered by activation: (i) Ral recruitment, c-Src activation, and subsequent Stat1 and Stat3 activation; (ii) Shc/Grb2 recruitment, Sos, Ras, and c-Raf activation, and subsequent MEK1/2 activation of ERK1/2; or (iii) Gab1 recruitment, Pk3K activation, subsequent generation of phosphatidylinositol-3,4,5-phosphate (Pl-3,4,5-P3), activation of PDK1/2 and then Akt that inactivate proapoptotic factors (see e.g. FIGS. 8, 9, 10, and 11).

As another example, an inhibitor of TNFα (a stimulator of EGFR expression in the airway epithelium) may be used to inhibit the EGFR signaling pathway. Similarly, the inhibitor can target components downstream of TNFα and achieve inhibition of EGFR signaling. EGFR can be activated through ligand-dependent and ligand-independent mechanisms, resulting in either autophosphorylation or trans-phosphorylation, respectively. Ligand-independent activators of EGFR signaling include oxidative stress (see e.g., Takeyama et al., Am. J. Physiol. Lung Cell Mol. Physiol., 280:L165-L172, 2001), ultraviolet and osmotic stress, stimulation of G-protein coupled-receptor by endothelin-1, lysophosphatidic acid and thrombin, m1 muscarinic acetylcholine receptor, and human growth hormone.

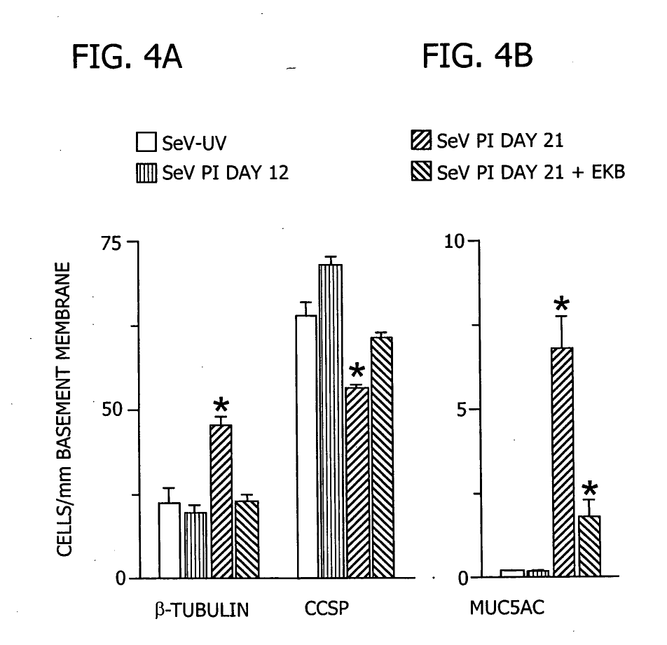

Inhibitors of IL-13 signaling include, for example, inhibitors targeting IL-4Rα, IL-13Rα1, ligands of IL4Rα, ligands of IL-13Rα1, and downstream IL-13 signaling components. (see e.g. FIG. 4s). As an example, IL-13 signaling can be inhibited by targeting the IL-4/IL-13 receptor. Alternatively, a factor or pathway that stimulates expression of the IL-4/IL-13 receptor (e.g., IL-13) is a target for an inhibitor of the IL-13 signaling pathway. Other activators of the IL-13 pathway include IL-4 and IL-9. As another example, an IL-13 signaling inhibitor can target (i) IRS1/2 recruitment, Grb2/Sos activation, Ras/c-Raf activation, and subsequent activation of ERK1/2 and Pk3K and/or (ii) the activation of Stat6, each of which contributes to upregulation of genes (e.g., CLCA and MUC) that promote cilia to goblet cell transdifferentiation (see e.g. FIG. 4s). Also, an IL-13 signaling inhibitor can target IRS1/2 recruitment, Pk3K activation, subsequent generation of phosphatidylinositol-3,4,5-phosphate (Pl-3,4,5-P3), activation of PDK1/2 and then Akt that inactivate proapoptotic factors (see e.g. FIGS. 8, 9, 10, and 11).

EGFR and IL-13 agonists can also be used to inhibit the respective signaling pathways. EGFR and IL-13 agonists are molecules which mimic interaction with receptors involved in the EGFR and IL-13 signaling pathway, respectively. Such may be analogs or fragments of signal molecules, or immunopeptides against ligand binding site epitopes of the receptors, or anti-idiotypic immunopeptides against particular immunopeptides which bind to receptor-interacting portions. Antagonists may take the form of proteins which compete for receptor binding but lack the ability to activate the receptor or binding molecules (e.g., immunopeptides). An example of an anti-IL-13-signaling agonists includes the recombinant soluble IL-13 receptor α2 Fc fusion protein, slL-13Rα2-Fc, which acts as a decoy receptor to specifically block IL-13 action (see e.g., Example 4).

Inhibitors of EGFR and IL-13 signaling generally include immunopeptides, small molecules acting as competitive or irreversible antagonsists, antisense oligonucleotides, and small interfering RNAs.

Immunopeptide Inhibitors

Immunopeptide inhibitors of EGFR and IL-13 signaling include, for example, polyclonal antibodies, monoclonal antibodies, and antibody fragments. Such antibodies can be produced by any appropriate method known to one skilled in the art; commercially produced antibodies may also be used.

Polyclonal antibodies may be readily generated by one of ordinary skill in the art from a variety of warm-blooded animals such as horses, cows, various fowl, rabbits, mice, or rats. Briefly, antigen is utilized to immunize the animal through intraperitoneal, intramuscular, intraocular, or subcutaneous injections, with an adjuvant such as Freund's complete or incomplete adjuvant. Following several booster immunizations, samples of serum are collected and tested for reactivity to the desired target. Particularly preferred polyclonal antisera will give a signal on one of these assays that is at least three times greater than background. Once the titer of the animal has reached a plateau in terms of its reactivity, larger quantities of antisera may be readily obtained either by weekly bleedings, or by exsanguinating the animal.

Monoclonal antibody (MAb) technology can be used to obtain MAbs capable of interfering with EGFR and/or IL-13 signaling pathways. Examples of antibodies that would function as an EGF antagonist include the neutralizing anti-EGFR monoclonal antibody C225 (Kawamoto et al. (1983) Proc. Nat'l. Acad. Sci. (USA) 80:1337-1341; Petit et al. (1997) J. Path. 151:1523-153, produced by ImClone Systems New York, N.Y.) and the anti-EGFR monoclonal antibody EMD55900 (also called Mab 425) (Merck, Darmstadt, Germany). Briefly, hybridomas are produced using spleen cells from mice immunized with antigens. The spleen cells of each immunized mouse are fused with mouse myeloma Sp 2/0 cells, for example using the polyethylene glycol fusion method of Galfre, G. and Milstein, C., Methods Enzymol., 73:3-46 (1981). Growth of hybridomas, selection in HAT medium, cloning and screening of clones against antigens are carried out using standard methodology (Galfre, G. and Milstein, C., Methods Enzymol., 73:3-46 (1981)). HAT-selected clones are injected into mice to produce large quantities of MAb in ascites as described by Galfre, G. and Milstein, C., Methods Enzymol., 73:3-46 (1981), which can be purified using protein A column chromatography (BioRad, Hercules, Calif.). MAbs are selected on the basis of their (a) specificity, (b) high binding affinity, (c) isotype, and (d) stability. MAbs can be screened or tested for specificity using any of a variety of standard techniques, including Western Blotting (Koren, E. et al., Biochim. Biophys. Acta 876:91-100 (1986)) and enzyme-linked immunosorbent assay (ELISA) (Koren et al., Biochim. Biophys. Acta 876:91-100 (1986)). These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

It may be desirable to produce and use functional antibody fragments, for example Fab, F(ab′)₂, Fc, and single chain Fv (scFv) fragments. These fragments will generally include hypervariable regions containing stretches of amino acid sequences known as complementarity determining regions, which are responsible for the antibody's specificity for one particular site on an antigen molecule. Proteolytic cleavage with papain produces two separate antigen binding fragments called Fab fragments which contain an intact light chain linked to an amino terminal portion of the contiguous heavy chain via by disulfide linkage. Proteolytic cleavage of a typical IgG molecule with papain produces a F(ab′)₂ fragment (Handbook of Experimental Immunology. Vol 1: Immunochemistry, Weir, D. M., Editor, Blackwell Scientific Publications, Oxford (1986)). Also, recombinant DNA methods permit the production and selection of recombinant immunoglobulin peptides which are single chain antigen-binding polypeptides known as scFv antibodies (Lowman et al. (1991) Biochemistry, 30, 10832-10838; Clackson et al. (1991) Nature 352, 624-628; and Cwirla et al. (1990) Proc. Natl. Acad. Sci. USA 87, 6378-6382).

Immunopeptide inhibitors can be administered in an amount of, for example, about 0.05 mg to about 2.5 mg per injection. As another example, immunopeptide inhibitors can be injected at a concentration of about 0.1 mg to about 1 mg per injection. Preferably, immunopeptide inhibitors are injected at a concentration of about 0.3 mg to about 0.5 mg per injection.

Small Molecule Inhibitors

Small molecule inhibitors of EGFR signaling include EGFR tyrosine kinase inhibitors. Many such EGFR tyrosine kinase inhibitors are known to the art and include PD153035, EKB-569, and AG1478 (4-(3-Chloroanilino)-6;7-dimethoxyquinazoline); non-phenolic tyrphostin analog EGFR inhibitor RG-14620; the EGFR receptor kinase inhibitors Tyrphostin 23 (RG-50810), Tyrphostin 25 (RG-50875), Tyrphostin 46, Tyrphostin 47 (RG-50864; AG-213), Tyrphostin 51 (BIOMOL Research Laboratories, Plymoth Meeting, Pa.; BioSource International, Camarillo Calif.); BIBX1522 (Boehringer Ingelheim, Inc., Ingelheim, Germany); CGP59326B (Novartis Corporation, Basel, Switzerland); 4-aminoquinazoline EGFR inhibitors (described in U.S. Pat. No. 5,760,041); substituted styrene compounds which can also be a naphthalene, an indane or a benzoxazine including nitrile and molononitrile compounds (described in U.S. Pat. No. 5,217,999); the inhibitors disclosed in U.S. Pat. No. 5,773,476; potato carboxypeptidase inhibitor (PCI), a 39-amino acid protease inhibitor with three disulfide bridges, (Blanco-Aparicio et al. (1998) J Biol Chem 273(20):12370-12377); bombesin antagonist RC-3095 (Szepeshazi et al. (1997) Proc Natl Acad Sci USA 94:10913-10918); pyridopyrimidines, pyrimidopyrimidines, pyrrolopyrimidines, such as CGP 59326, CGP 60261 and CGP 62706, and pyrazolopyrimidines (Strawn and Shawver (1998) Exp.-Opin. Invest. Drugs 7(4), 553-573); 4-(phenylamino)-7H-pyrrolo[2,3-d] pyrimidines (Traxier et al. (1996) J. Med. Chem 39:2285-2292); curcumin (Korutla et al. (1994) Biochim Biophys Acta 1224:597-600); (Laxmin arayana (1995), Carcinogen 16:1741-1745); etc.

Small molecule inhibitors of EGFR signaling also include the G-protein uncoupler Suramin Sodium (BIOMOL Research Laboratories, Plymoth Meeting, Pa.; BioSource International, Camarillo Calif.).

Small molecule inhibitors of IL-13 signaling include those that target downstream signaling such as PD98059 (targeting MEK 1/2→ERK 1/2) and LY294002 (targeting Pk3K→AKT).

Small molecule inhibitors can be administered in an amount of about 0.1 μg to about 100 mg per kg weight of subject per administration. Those of skill will readily appreciate that dose levels can vary as a function of the specific compound, the severity of the symptoms and the susceptibility of the subject to side effects, as discussed more fully below.

Antisense Inhibitors

Antisense oligodeoxynucleotides inhibit gene expression on a highly selective and specific manner by hybridizing to complementary mRNA and decreasing protein expression.

Antisense oligonucleotide EGFR signaling inhibitors and methods for their production include, for example, those described in Kronmiller et al. (1991) Dev Biol. 147(2), 485-8; Hu et al. (1992) Int J Dev Biol. 36(4), 505-16; Roy and Harris (1994) Molecular Endocrinology 8, 1175-1181; Casamassimi et al. (2000) Ann-Oncol 11(3), 319-325; Normanno et al. (1996) Cancer Detection and Prevention 20(5); He et al. (2000) World J Gastroentero 6(5), 747-749; Riedel et al., Int J Oncol (2002) 21,11-16; Zeng et al. (2002) J Exp Ther Oncol 2(3), 174-186; Deng et al. (2003) Di Yi Jun Yi Da Xue Xue Bao 23(9), 877-81; Li et al. (2002) Clin Cancer Res 8, 3570-3578. Antisense oligonucleotide EGFR signaling inhibitors also include those produced by methods similar to those above. The safety and efficacy of EGFR antisense gene therapy is discussed in Zeng et al. (2002) J Exp Ther Oncol 2(3), 174-186.

Antisense oligonucleotide IL-13 signaling inhibitors and methods for their production include, for example, those described in Mousavi et al. (2004) Iran. Biomed. J. 8(4), 185-191.

Antisense oligonucleotides can be administered via intravitreous injection at a concentration of about 10 μg/day to about 3 mg/day. For example, administered dosage can be about 30 μg/day to about 300 μg/day. As another example, antisense oligonucleotide can be administered at about 100 μg/day. Administration of antisense oligonucleotides can occur as a single event or over a time course of treatment. For example, antisense oligonucleotides can be injected daily, weekly, bi-weekly, or monthly. Time course of treatment can be from about a week to about a year or more. In one example, antisense oligonucleotides are injected daily for one month. In another example, antisense oligonucleotides are injected weekly for about 10 weeks. In a further example, antisense oligonucleotides are injected every 6 weeks for 48 weeks.

RNA Interference

The EGFR and IL-13 signaling pathways can be down-regulated by RNA interference by administering to the patient a therapeutically effective amount of small interfering RNAs (siRNA) specific for components of these pathways, such as EGF, EGFR, IL-13, IL-4Rα, or IL-13Rα1. siRNA is commercially available from sources such as Ambion (Austin, Tex.). The siRNA can be administered to the subject by any means suitable for delivering the siRNA to the cells of the tissue at or near the area of epithelial hyperplasia and metaplasia and/or hypersecretion. For example, the siRNA can be administered by gene gun, electroporation, or by other suitable parenteral or enteral administration routes, such as intravitreous injection.

RNA interference is the process by which double stranded RNA (dsRNA) specifically suppresses the expression of a gene bearing its complementary sequence. Suppression of the gene inhibits the production of the corresponding protein. Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. Preferably, the siRNA comprises short double-stranded RNA from about 17 nucleotides to about 29 nucleotides in length, preferably from about 19 to about 25 nucleotides in length, that are targeted to the target mRNA.

As an example, an effective amount of the siRNA can be an amount sufficient to cause RNAi-mediated degradation of the target mRNA, or an amount sufficient to inhibit the EGFR or IL-13 signaling pathways in a subject. One skilled in the art can readily determine an effective amount of the siRNA of the invention to be administered to a given subject by taking into account factors such as the size and weight of the subject; the extent of the neovascularization or disease penetration; the age, health and sex of the subject; the route of administration; and whether the administration is regional or systemic. Generally, an effective amount of siRNA comprises an intercellular concentration at or near the epithelial hyperplasia and metaplasia site of from about 1 nanomolar (nM) to about 100 nM, preferably from about 2 nM to about 50 nM, more preferably from about 2.5 nM to about 10 nM. It is contemplated that greater or lesser amounts of siRNA can be administered.

The siRNA can be targeted to any stretch of approximately 19-25 contiguous nucleotides in any of the mRNA target sequences. Searches of the human genome database (BLAST) can be carried out to ensure that selected siRNA sequence will not target other gene transcripts. Techniques for selecting target sequences for siRNA are given, for example, in Elbashir et al. ((2001) Nature 411, 494-498). Thus, the sense strand of the present siRNA comprises a nucleotide sequence identical to any contiguous stretch of about 19 to about 25 nucleotides in the target mRNA of EGF, EGFR, IL-13, IL-4Rα, or IL-13Rα1. Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon.

Dosage and Time Course of Treatment

When used in the treatments described herein, a therapeutically effective amount of one of the compounds of the present invention may be employed in pure form or, where such forms exist, in pharmaceutically acceptable salt form and with or without a pharmaceutically acceptable excipient. For example, the compounds of the invention can be administered in a sufficient amount to inhibit EGFR signaling and IL-13 signaling or reduce the formation of products resulting from the EGFR and IL-13 signaling cascade at a reasonable benefit/risk ratio applicable to any medical treatment. Specific dosages for each type of inhibitor are discussed more fully above. It will be understood, however, that the total daily usage of the compounds and compositions of the present invention will be decided by the attending physician within the scope of sound medical judgment.

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose.

Administration of the inhibitors of EGFR and IL-13 signaling can occur as a single event or over a time course of treatment. For example, inhibitors can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

According to the methods presented herein, prophylactic and therapeutic treatment of inflammatory disorders characterized by EGFR activation, IL-13 expression, and goblet cell metaplasia can be effected through blockade, reduction, or down-regulation of EGFR and IL-13 signaling through administration of inhibitors of these pathways.

Therapeutic Administration

The EGFR and IL-13 signaling inhibitors can be used therapeutically either as exogenous materials or as endogenous materials. Exogenous agents are those produced or manufactured outside of the body and administered to the body. Endogenous agents are those produced or manufactured inside the body by some type of device (biologic or other) for delivery to within or to other organs in the body.

Exogenous Therapy

A safe and effective amount of EGFR and IL-13 signaling inhibitors is, for example, that amount that would cause the desired therapeutic effect in a patient while minimizing undesired side effects. The dosage regimen will be determined by skilled clinicians, based on factors such as the exact nature of the condition being treated, the severity of the condition, the age and general physical condition of the patient, and so on.

The compositions of the present invention will include one or more EGFR and IL-13 signaling inhibitors and a pharmaceutically acceptable vehicle for said compound(s). Various types of vehicles may be used. The vehicles can be aqueous in nature. The compounds can also be readily incorporated into other types of compositions, such as suspensions, viscous or semi-viscous gels or other types of solid or semi-solid compositions. Suspensions may be preferred for agents which are relatively insoluble in water. The compositions of the present invention may also include various other ingredients, such as buffers, preservatives, co-solvents and viscosity building agents.

The EGFR and IL-13 signaling inhibitors may be contained in various types of pharmaceutical compositions, in accordance with formulation techniques known to those skilled in the art. The specific type of formulation selected will depend on various factors, such as EGFR and IL-13 signaling inhibitors being used, the dosage frequency, and the location being treated. For example, the agents may be included in solutions, suspensions and other dosage forms adapted for topical application to the involved tissues, such as tissue irrigating solutions, or injection to the involved tissues. An appropriate buffer system (e.g., sodium phosphate, sodium acetate or sodium borate) may be added to prevent pH drift under storage conditions.

Preservatives are thus generally required to prevent microbial contamination during use. Examples of suitable preservatives include: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from about 0.001 to about 1.0 percent by weight, based on the total weight of the composition (wt. %).

Some of the EGFR and IL-13 signaling inhibitors may have limited solubility in water and therefore may require a surfactant or other appropriate co-solvent in the composition. Such co-solvents include: polyethoxylated castor oils, Polysorbate 20, 60 and 80; Pluronic Registered TM F-68, F-84 and P-103 (BASF Corp., Parsippany N.J., USA); cyclodextrin; or other agents known to those skilled in the art. Such co-solvents are typically employed at a level of from about 0.01 to about 2 wt. %.

Physiologically balanced irrigating solutions can be used as pharmaceutical vehicles for the EGFR and IL-13 signaling inhibitors. As used herein, the term “physiologically balanced irrigating solution” means a solution which is adapted to maintain the physical structure and function of tissues during invasive or noninvasive medical procedures. This type of solution will typically contain electrolytes, such as sodium, potassium, calcium, magnesium, and/or chloride; an energy source, such as dextrose; and a buffer to maintain the pH of the solution at or near physiological levels. Various solutions of this type are known (e.g., Lactated Ringers Solution). BSS Registered TM Sterile Irrigating Solution and BSS Plus Registered TM Sterile Intraocular Irrigating Solution (Alcon Laboratories, Inc., Fort Worth, Tex., USA) are examples of physiologically balanced intraocular irrigating solutions.

Viscosity greater than that of simple aqueous solutions may be desirable to increase tissue absorption of the active compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose or other agents known to those skilled in the art. Such agents are typically employed at a level of from about 0.01 to about 2 wt. %.

The compositions of the invention can be packaged in multidose form.

Endogenous Therapy

The principles of gene therapy for the production of therapeutic products can be used to deliver EGFR and IL-13 signaling inhibitors. In clinical settings, the gene delivery systems for therapeutic EGFR and IL-13 signaling inhibitors can be introduced into a patient (or non-human animal) by any of a number of methods, each of which is known in the art. For example EGFR and IL-13 antisense nucleic acid signaling inhibitors can be introduced via delivery vehicles (termed vectors) that can be non-pathogenic viral variants (e.g., replication-defective murine retroviral vectors and adeno-associated viral vectors)), lipid vesicles (e.g., liposomes, lipofectins, and cytofectins), carbohydrate and/or other chemical conjugates of nucleotide sequences encoding the therapeutic protein or substance. As another example, vectors can be introduced into the body's cells by physical (e.g., microinjection, electroporation, and pneumatic “gene gun”), chemical, or cellular receptor (e.g., receptor-based endocytosis) mediated uptake. Once within the cells, the nucleotide sequences can be made to produce the therapeutic substance within the cellular (episomal) or nuclear (nucleus) environments. Episomes usually produce the desired product for limited periods whereas nuclear incorporated nucleotide sequences can produce the therapeutic product for extended periods including permanently.

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, and the transfected cells are expanded in number and then reimplanted in the patient. In in vitro gene transfer, the transformed cells are cells growing in culture, such as tissue culture cells, and not particular cells from a particular patient. These “laboratory cells” are transfected, and the transfected cells are selected and expanded for either implantation into a patient or for other uses. In vivo gene transfer involves introducing the DNA into the cells of the patient when the cells are within the patient. In vivo gene transfer also involves introducing the DNA specifically into the ocular endothelial cells of the patient using gene therapy vectors containing endothelial specific promoters. All three of the broad-based categories described above may be used to achieve gene transfer in vivo, ex vivo, and in vitro.

Gene therapy also contemplates the production of a protein or polypeptide where the cell has been transformed with a genetic sequence that turns off the naturally occurring gene encoding the protein, i.e., endogenous gene-activation techniques.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Methods Used in the Following Examples

While certain of the methods used in the Examples described below are contained within the Examples themselves, additional techniques were also employed and are described immediately below.

PROLIFERATION MARKERS. For BrdU immunostaining, mice received BrdU (100 mg/kg) intraperitoneally at 48 h, 24 h and 4 h prior to euthanasia. BrdU was detected with an anti-BrdU staining kit (Zymed Laboratories, Inc., San Francisco, Calif.) according to the manufacturer's protocol. Ki67 immunostaining was performed with anti-Ki67 Ab (Novocastra Laboratories Ltd, Newcastle, UK) using the same protocol as for EGFR immunostaining except for pretreatment of heat-induced antigen retrieval using Antigen Unmasking Solution (Vector Laboratories). PCNA staining was performed with biotinylated anti-mouse PCNA Ab (DAKO Corporation, Carpinteria, Calif.) using the ABC method (Vector Laboratories).

AIRWAY EPITHELIAL CELL CULTURE AND TREATMENT. Primary air-liquid interface cultures of mouse tracheal epithelial cells (mTECs) were established as described previously (You, et al. (2002) Am. J. Physiol. Lung Cell Mol. Physiol. 283:L1315-1321). Human airway epithelial cell cultures were established from tracheobronchial specimens harvested from lung explants of COPD patients undergoing transplantation and from lung transplant donors without lung disease using the same culture conditions. In all cases, cells were grown in basic medium (DMEM/Ham's F-12 with 30 mM HEPES, 4 mM L-glutamine, 3.5 mM NaHCO3, 0.01% Fungizone, and penicillin/streptomycin) supplemented with 10 μg/ml insulin, 10 μg/ml transferrin, 0.1 μg/ml cholera toxin, 25 ng/ml EGF (Bectin Dickinson, Bedford, Mass.), 30 μg/ml bovine pituitary extract, and 5% FBS in the upper and lower compartments. After the cells developed transmembrane electrical resistance>1000 Ohm·cm², the air-liquid-interface condition was established by washing the membrane with PBS and changing the medium in the lower compartment to basic medium supplemented with 2% NuSerum (BD BioSciences, San Diego, Calif.). For EGFR stimulation, cells were incubated in basic medium for 24 h and then in basic medium containing EGF (1-100 ng/ml, Upstate Biotechnology, Lake placid, N.Y.) added to the upper and/or lower compartments for 10 min at 37° C. EGFR signaling inhibitors or vehicle control (0.1% DMSO) were added to the lower compartments on a daily basis for long-term experiments or for 1.5 to 6 h to the upper and lower compartments for short-term experiments. EGFR tyrosine kinase inhibitor PD153035, MEK1/2 inhibitor PD98059, EGFR tyrosine kinase inhibitor AG1478, and Pk3K inhibitor LY294002 were from Calbiochem (La Jolla, Calif.), and z-Val-Ala-Asp fluoromethylketone (z-VAD-fmk) was from Enzyme Systems Products (Livermore, Calif.). Recombinant human or mouse IL-13 from Preprotech (Rocky Hill, N.J.) was added to upper and lower compartments at 24 h before air-liquid-interface conditions and was maintained in the lower compartment throughout the experiment.

IMMUNOCYTOCHEMISTRY. Cultured cells were washed twice with PBS at 4° C., fixed in 4% paraformaldehyde for 10 min. at 25° C., washed with PBS, and permeabilized with ethanol:acetic acid (2:1, vol/vol) for 5 min at −20° C. for TUNEL reaction or with 0.2% Triton-X for 5 min at 25° C. for immunostaining. Permeabilized cells were then washed with PBS and subjected to the TUNEL reaction (Intergen, Purchase, N.Y.) or blocked with 2% fish gel 1 h at 25° C. and incubated with rabbit anti-active caspase 3 (BD Biosciences, San Diego, Calif.), rabbit anti-EGFR (Santa Cruz Biotechnology, Santa Cruz, Calif.), rabbit anti-p-EGFR (Cell Signaling Technology, Inc., Beverly, Mass.), mouse anti-β-tubulin-IV or rabbit anti-β-tubulin (Sigma, St. Louis, Mo.) antibodies overnight at 4° C. Primary antibody binding was detected with goat anti-mouse or donkey anti-rabbit FITC or CY3 secondary antibody. Cells were counterstained with 4 μg/ml Hoechst 33258 (Molecular Probes, Eugene, Oreg.) to check nuclear morphology, and then imaged as described above.

FLOW CYTOMETRY. Mouse tracheal epithelial cells were cultured as above and removed from Transwell culture using cell dissociation solution (Sigma, St. Louis, Mo.) containing 0.25% Trypsin and 0.1% EDTA. Cells were washed with HBSS containing 0.2% BSA and incubated with 5 μg/ml JC-1 (Molecular Probes, Eugene, Oreg.) for 15 min at 25° C. Cells with mitochondrial membrane depolarization were detected by a shift from low to high emission in green fluorescence (FL1) using a FACSCalibur flow cytometer and CellQuest software (Becton Dickinson, Mountain View, Calif.).

ELECTRON MICROSCOPY. Cells on membranes were prepared for transmission electron microscopy (TEM) as previously described (You, et al. (2004) Am. J. Physiol. Lung Cell. Mol. Physiol. 286:L650-657). In brief, samples were fixed with 2.5% glutaraldehyde and stained with 1.25% osmium tetroxide. Cells were counterstained with 2.0% tannic acid, blocked for sectioning, and imaged on a Zeiss 902 model microscope.

STATISTICAL ANALYSIS. Values for histochemistry of mouse tissues were analyzed using a one-way analysis of variance (ANOVA) for a factorial experimental design. If significance was achieved by one-way analysis, post-ANOVA comparison of means was performed using Scheffe's F test.

### Example 1

### Persistent Activation of EGFR on Ciliated Epithelial Cells

EGFR behavior in mouse airway epithelium was assessed in mice inoculated with mouse parainfluenza virus (Sendai virus; SeV), as common human paramyxoviruses (e.g., respiratory syncitial virus or metapneumovirus) generally replicate poorly in mice. In this model system, inoculation results in replication with high efficiency in the bronchiolar mucosa with consequent induction of immune-response gene expression, immune cell infiltration, and damage of the epithelium (Walter et al. (2001) J. Exp. Med. 193, 339-352). This host response allows for complete clearance of SeV by 10-12 days after inoculation. (Walter, et al. (2002). J. Clin. Invest. 110:165-175; Tyner, et al. (2005) Nat. Med. 11:1180-1187) The injury is followed by epithelial repair and restoration of normal airway architecture in some mouse strains, but can be followed by long-term (likely permanent) goblet cell metaplasia in C57BU6J mice that appears about 21 days after inoculation (Walter et al. (2002) J. Clin. Invest. 110,165-175).

C57BL/6J and Balb/cJ mice were obtained from The Jackson Laboratory (Bar Harbor, Me.) and were maintained and monitored under pathogen-free conditions for study at 7 wk of age as described previously (Walter et al. (2001); Walter et al. (2002); Tyner et al. (2006) J. Clin. Invest. 116:309-321). SeV (Fushimi Strain 52) was grown in embryonated hen eggs and harvested to provide a viral stock solution such that 5000 EID50 (50% egg infectious dose) was equivalent to 2×10⁵ PFU (plaque-forming units). This inoculum or an equivalent amount of UV-inactivated SeV was delivered intranasally in 30 μl PBS under ketamine/xylazine anesthesia. Under these conditions, viral tissue levels are maximal at days 3-5 after infection and viral clearance is complete by day 12 (Walter et al. (2001); Walter et al. (2002); Tyner et al. (2006) J. Clin. Invest. 116:309-321). Sentinel mice and experimental control mice were handled identically to inoculated mice and exhibited no serologic or histologic evidence of exposure to 11 rodent pathogens (including SeV). For EGFR blockade, mice were treated with EKB-569 (obtained from Lee Greenberger, Wyeth Ayerst Pharmaceuticals, Pearl River, N.Y.; 20 mg/kg in pH 2.0 water given by gavage) or vehicle control given daily from post-infection days 10-21. For IL-13 blockade, mice were given subcutaneous injections of soluble murine IL-13Rα2 fused to Fc (slL Example 2: EGFR inhibition decreases aspects of epithelial remodeling 13Rα2-Fc; obtained from Deborah Donaldson, Wyeth Ayerst; 200 μg/mouse in PBS) or control Fc on days 12, 14, 17, and 20 post-infection (Donaldson (1998) J. Immunol. 161, 2317).

For whole lung analysis, the left lobe of mouse lung was homogenized in RIPA buffer Page 16 (1% NP-40, 0.5% sodium deoxycholate, 0.1% SDS in PBS) containing phosphatase inhibitor cocktail (Sigma). Tracheal tissue and mTECs were collected in cell lysis buffer containing 20 mM Tris-HCl, 150 mM NaCl, 1 mM EDTA, 1 mM EGTA, 1% Triton-X 100, 1.0 μg/ml leupeptin, 10 μg/ml aprotinin, 0.2 mM phenylmethylsulfonyl fluoride, 1 mM sodium orthovanadate, 0.1 mM sodium fluoride, 2.5 mM sodium pyrophosphate, and 1 mM β-glycerophosphate. Cell lysates were cleared by centrifugation, and supernatant proteins were separated on 4-15% gradient SDS-PAGE and transferred to PVDF membranes (Millipore, Bedford, Mass.). The membranes were blotted against antibodies to EGFR, phospho-EGFR, phospho- ERK1/2, activated caspase 3, phosphor-Stat6 (Cell Signaling Technology, Beverly, Mass.), phospho-Akt (BD Biosciences, San Diego, Calif.), caspase 9 (Stressgen, San Diego, Calif.), and β-actin (Chemicon, Temecula, Calif.). Primary antibody binding was detected with secondary antibodies conjugated to horseradish peroxidase and enhanced chemiluminescence (Amersham Pharmacia Biotech, Buckinghamshire, UK).

Mouse lung was fixed by intraracheal instillation of 4% paraformaldehyde at 25-cm H₂O pressure. After overnight fixation at 4° C., tissue was embedded in paraffin, and cut into 3-μm thick sections for hematoxylin/eosin or immunostaining.

For human tissue, asthmatic subjects (with and without glucocorticoid treatment) and healthy control subjects were recruited, characterized, and subjected to endobronchial biopsy as described previously (Walter et al. (2001); Sampath et al. (1999) J. Clin. Invest. 103:1353-1361; Taguchi (1998) J. Exp. Med. 187, 1927-1940). In brief, for asthmatic subjects, endobronchial biopsies were taken after subjects were treated with inhaled fluticasone proprionate (1760 μg/d) for 30 days and then after fluticasone was discontinued for 6 wk or until peak expiratory flow had decreased by 25% and forced expiratory volume in 1 sec by 15%. For all subjects, there was no history of respiratory infection for the previous 3 months. Endobronchial biopsies were washed with PBS and incubated with 10% neutral buffered formalin for 18 h at 25° C. followed by histochemistry as described above. In addition, lung tissue samples from COPD patients that were undergoing lung resection or transplantation were obtained and processed as described above.

For immunostaining, tissue sections were deparaffinized, rehydrated in graded alcohol and encircled with a hydrophobic film (ImmEdge PEN, Vector laboratories, Burlingame, Calif.). For antigen retrieval, sections were digested with Proteinase K (Sigma, St. Louis, Mo.) at a final concentration of 40 μg/ml in PBS for 5 min and then treated in 3% hydrogen peroxide in distilled water for 10 min to quench endogenous peroxidase activity. Nonspecific protein binding was blocked with 3% BSA and 2% goat serum in Tris-buffered saline (pH 8) with 0.2% Tween 20 (TBST) for 1 h. Primary antibodies were diluted in blocking buffer and incubated overnight at 4° C. at a final concentration of 0.05 or 0.1 μg/ml for human and mouse tissue sections, respectively. EGFR was detected using rabbit-antihuman EGFR antibody SC-03 from Santa Cruz Biotechnology (Santa Cruz, Calif.) directed against amino acid residues 1005-1016 that are identical to corresponding sequence in murine EGFR. Phosphorylated EGFR (p-EGFR) was detected using rabbit anti-phospho-EGFR (Tyr845) antibody #2231 from Cell Signaling Technology Inc. (Beverly, Mass.) directed against phosphorylated Tyr⁸⁴⁵. For this antibody, final concentrations of 0.16 and 0.32 μg/ml were used for human and mouse tissues, respectively. Ciliated, Clara, and goblet cells were identified using mouse anti-β-tubulin-IV mAb (Sigma), goat anti-Clara cell secretory protein (CCSP) antibody (Santa Cruz Biotechnology), and mouse anti-human MUC5AC mAb 45M1 (Lab Vision Corp., Fremont, Calif.), respectively. To verify specificity, sections were also incubated with primary antibodies that were pre-absorbed with 10-fold excess of peptide antigen or with non-immune rabbit IgG (Santa Cruz). After primary antibody binding, sections were washed with TBST and then incubated with biotinylated goat anti-rabbit IgG (2 μg/ml). Signals were amplified with the Elite ABC method and 3,3′-diaminobenzidine chromogen according to the manufacturer's protocols (Vector Laboratories). Sections were counterstained with hematoxylin, dehydrated, and mounted with Cytoseal 60 (Stephens Scientific, Riverdale, N.J.). Immunofluorescence was performed in the same manner as immunostaining for light microscopy except that tissues were frozen in Tissue-Tek OCT (Sakura Finetek, Torrance, Calif.), sections were blocked with 2% donkey serum (Jackson ImmunoResearch Labs, West Grove, Pa.), primary antibody binding was detected using CY-3- or FITC-conjugated antibodies (Jackson lmmunoResearch Labs) for 30 min at 25° C., and sections were counterstained with Hoechst dye 33432 (Molecular Probes, Eugene, Oreg.). Sections were imaged with light or immunofluorescent microscopy (Olympus Model BX-51) interfaced to a digital photomicrography system (Optronix CCD Camera and Magnafire v2 software). Reporter was quantified by counting ciliated cells in pulmonary airways per mm of basement membrane with analysis performed on a Macintosh computer using the public domain NIH Image program (developed at the U.S. National Institutes of Health and available on the Internet at rsb.info.nih.gov/nih-image) as described previously (Walter et al. (2001); Walter et al. (2002); Sampath et al. (1999)). Confocal microscopy was performed using a Zeiss laser scanning system with LSM-510 software (Zeiss, Thornwood, N.Y.).

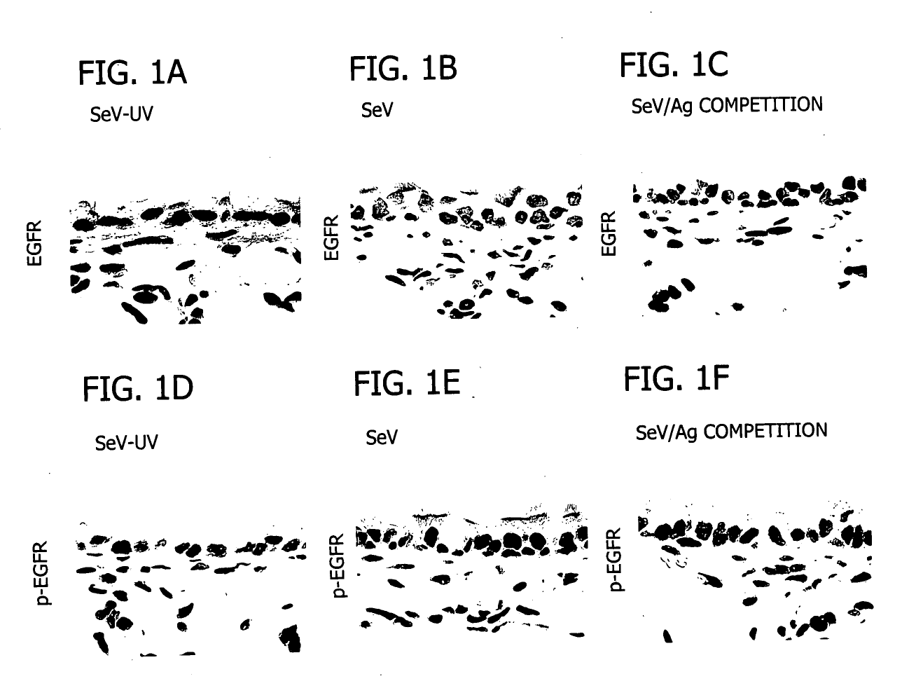

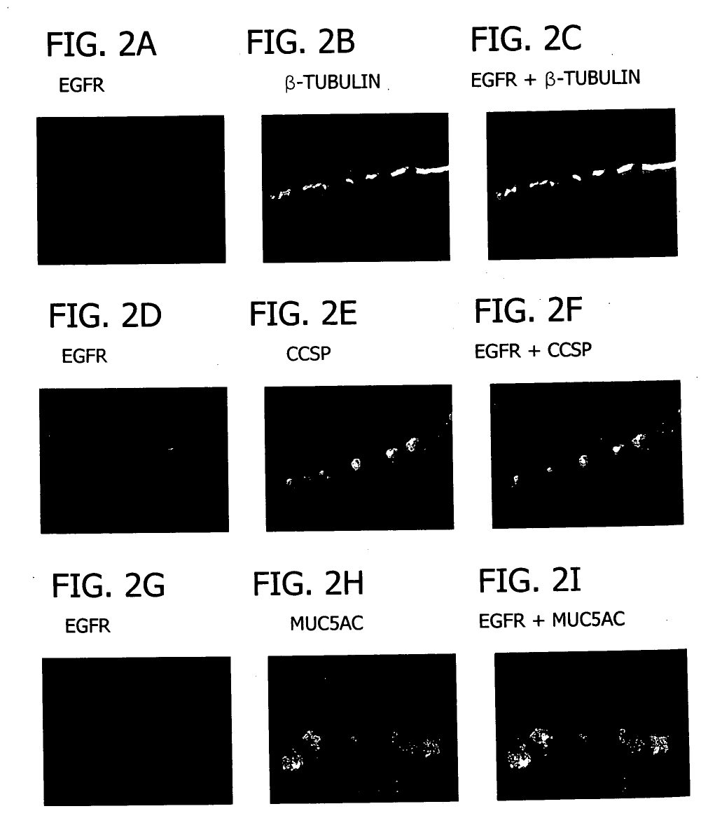

Results from Western blot analysis indicated that anti-EGFR and anti-phospho-EGFR antibodies specifically recognized the receptor in airway tissue samples (data not shown). Results from immunostaining of airway tissue with anti-EGFR antibody indicated that EGFR expression was predominantly localized to the apical membrane of ciliated epithelial cells, although other cell types (e.g., basal cells and airway smooth muscle cells) were also weakly immunostained (FIG. 1). No significant difference was observed in the pattern or the level of anti-EGFR immunostaining between mice infected with SeV versus control mice that were inoculated with SeV-UV. By contrast, immunostaining for phospho-EGFR (using anti-phospho-EGFR antibody that recognizes phosphorylated Tyr⁸⁴⁵) indicated that levels of activated EGFR were persistently increased at about 21 days after inoculation with SeV compared to uninoculated or SeV-UV inoculated control mice. Similar to the pattern for EGFR expression, phospho-EGFR was also localized mainly to the apical surface of ciliated epithelial cells, but in this case, apical cell staining was also accompanied by corresponding nuclear staining in this same ciliated cell population (FIG. 1). Other cell types (e.g., basal cells) were also weakly immunostained in nuclear and cytosolic locations. These findings are consistent with reports of nuclear translocation of activated EGFR (Lin et al. (2001) Nature Cell Biol. 3:802-808). This pattern of immunostaining indicated that the initial EGFR antibody recognizes predominantly the unphosphorylated receptor. For both antibodies, immunostaining was completely abolished by preabsorbtion with corresponding antigen. In addition, normal rabbit IgG was used as a negative isotype control and showed no significant signal above background. Results from double-labeling and immunofluorescence detected by laser scanning confocal microscopy indicated that EGFR colocalized with a marker for ciliated epithelial cells (i.e., β-tubulin) but not with markers for Clara cells (i.e., CCSP) or goblet cells (i.e., MUC5AC) in mouse airways (FIG. 2). This is in agreement with EGFR expression localized predominantly to ciliated epithelial cells.

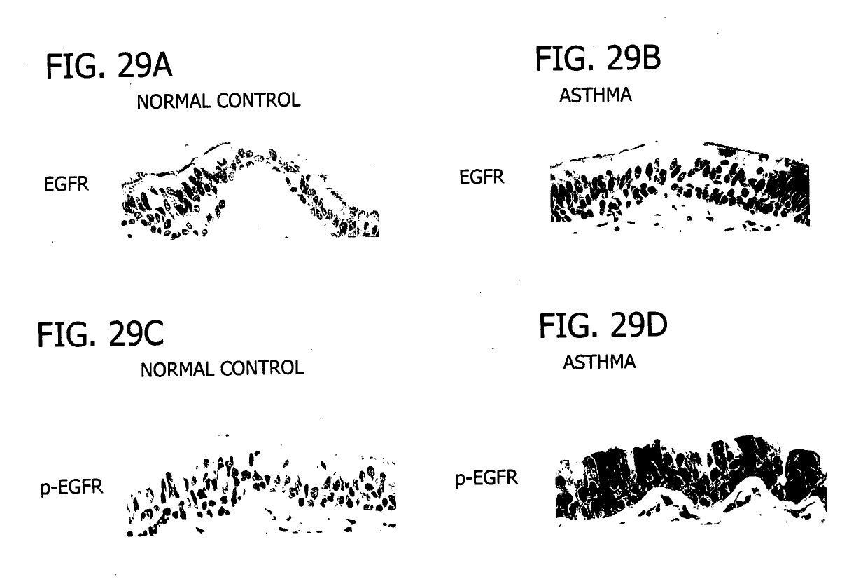

The pattern of EGFR immunostaining found in mice was similar to the one in human subjects. In particular, EGFR expression was also localized to the apical cell membrane of ciliated epithelial cells in normal and asthmatic subjects, and phospho-EGFR was increased in asthmatic subjects that also manifest goblet cell metaplasia (FIG. 29). Expression of phospho-EGFR was similarly localized to the apical portion of ciliated epithelial cells, and expression was accompanied by corresponding nuclear staining in the same ciliated cells. Additional albeit weaker phospho- EGFR immunostaining was also present on basal cells in both normal and asthmatic subjects.

### Example 2

### Functional Role of EGFR Signaling on Ciliated Epithelial Cells

To define a functional role for persistent EGFR signaling on ciliated epithelial cells, lung sections were subjected to immunostaining with markers for ciliated epithelial cells, Clara cells, and goblet cells. Tissue preparation and immunostaining were as described above.

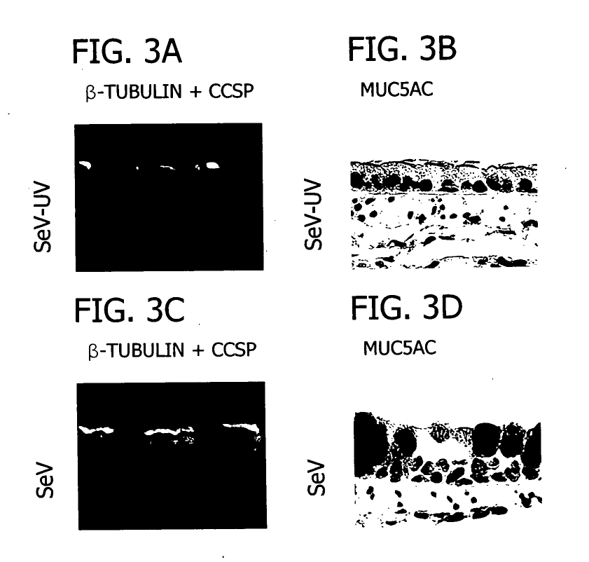

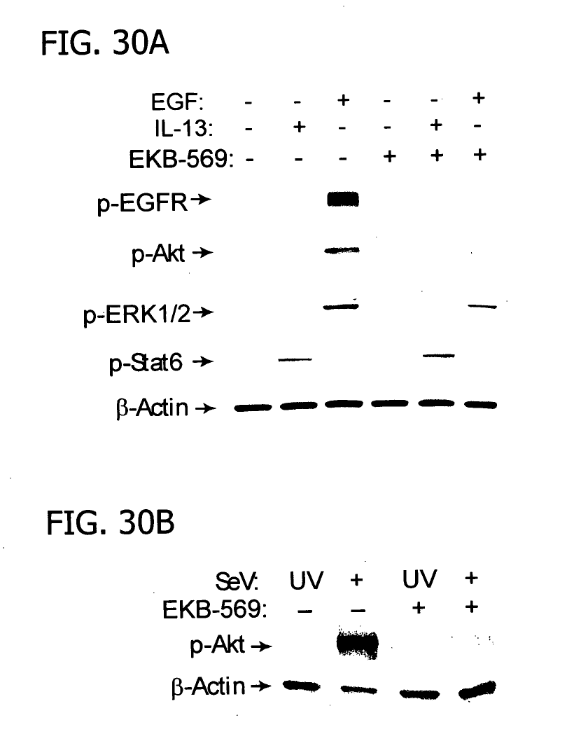

Quantitative analysis of cell types found in the airway epithelium indicated that SeV-infected mice developed increases in ciliated and goblet cells and concomitant decreases in Clara cells at day 21 (but not by day 12) after inoculation compared to control mice inoculated with SeV-UV (FIGS. 3-4). It was next determined whether EGFR signaling is necessary for these observed changes in epithelial architecture. Using an orally administered, irreversible EGFR inhibitor, EKB-569, to treat mice daily from postinfection day 10 through 21, full inhibition of Akt activation was achieved in whole mouse lung at day 21 post-infection (FIG. 30), indicating that EGFR pro-survival signaling was effectively blocked under these conditions. For these studies, a new irreversible EGFR inhibitor (EKB-569) was used that selectively inhibits EGFR signaling in airway epithelial cells in vitro (FIG. 30). To achieve blockade in vivo, EKB-569 was administered orally each day from post-inoculation day 10 (so as not to interfere with viral clearance or epithelial repair) through day 21 (when the remodeling response developed). Under these treatment conditions, EKB-569 also blocked EGFR signaling in vivo (FIG. 30).

EGFR inhibitor treatment was also observed to help correct all three aspects of epithelial remodeling. Specifically, complete blockade of ciliated cell increases and Clara cell decreases, and partial but significant inhibition of goblet cell metaplasia, was observed (FIG. 6). EKB-569 treatment had no effect on the total number of airway epithelial cells (cytokeratin-staining cells were 133±3 after vehicle and 137±5 per mm basement membrane after drug treatment on post-inoculation day 21), consistent with compensatory changes in other epithelial cell (e.g., basal and Clara cell) populations. Based on immunohistochemical data showing localization of EGFR and β-tubulin expression together in the same ciliated cell population, it was expected that EGFR blockade might influence ciliated cell hyperplasia. However, the effect of interrupting EGFR signals on Clara cell or goblet cell levels was unexpected based on the relative absence of activated EGFR expression on either of these cell types.

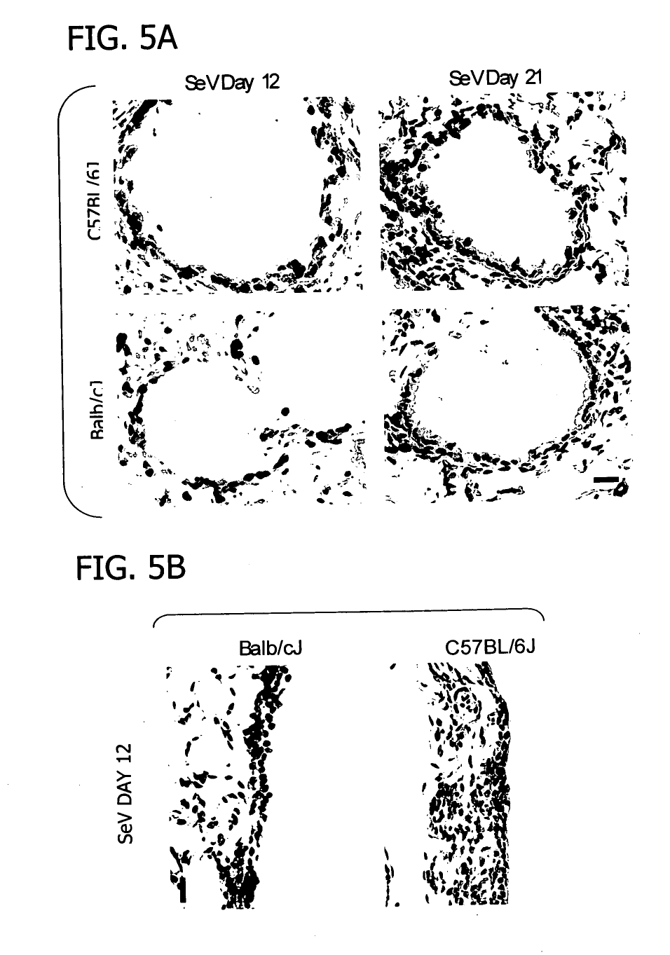

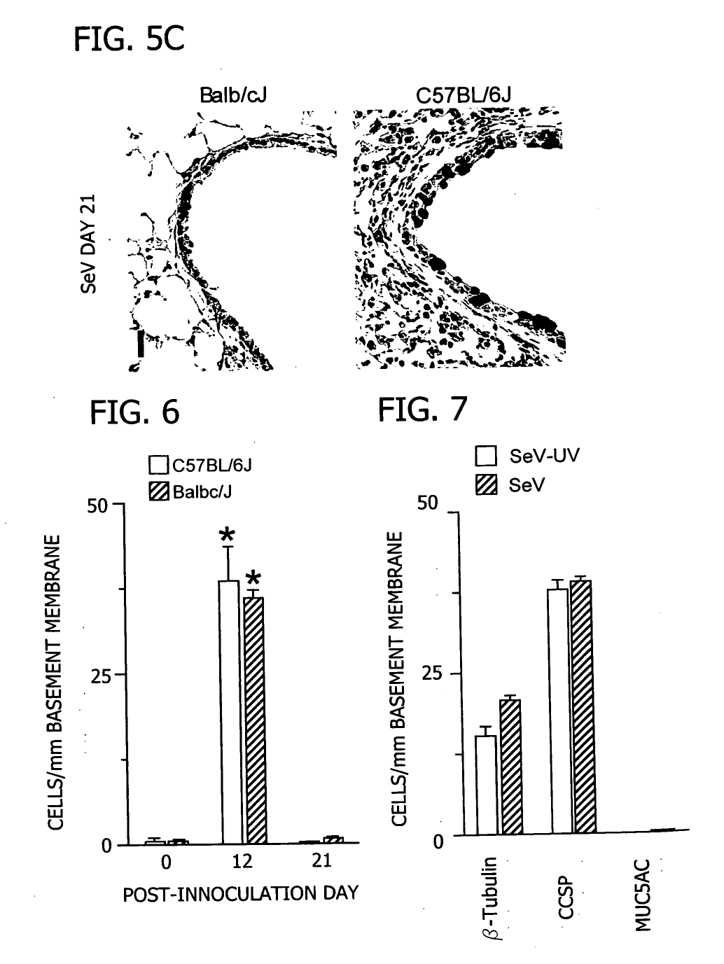

Further experiments were designed to better understand the mechanisms underlying the role of EGFR in chronic epithelial remodeling. Epithelial hyperplasia could either be a result of increased proliferation or decreased cell death. As such, evidence of increased proliferation in mice with epithelial remodeling was sought. As noted previously, there is transient epithelial proliferation (marked by BrdU labeling) during days 5-12 after infection (FIGS. 5A and 6; (Look et al. (2001) Am. J. Pathol. 159, 2055-2069)). The same pattern of immunostaining was found for Ki-67 and PCNA proliferation markers (data not shown). This proliferative response likely allows for replacement of host cells that suffer direct cytopathic effects and immune-mediated cell death in the wake of viral replication (Tyner, J. W. et al. (2006) J. Clin. Invest. 116:309-321). Not surprisingly, this repair phase is accompanied by EGFR activation in epithelial cells (generally basal cells) as well as subepithelial (likely immune) cells (FIG. 5B). However, by post-inoculation day 21, there was no longer evidence of an ongoing proliferative response, since cellular proliferation was no different than non-inoculated control mice (FIGS. 3A and 3B). Moreover, this replacement phase (marked by BrdU uptake and EGFR activation in the basal cell compartment) was the same in a strain of mice (Balbc/J) that does not develop long-term epithelial remodeling (FIGS. 5A-5C, 6, and 7). Thus, this transient proliferative response could not account for the subsequent long-term remodeling that was found only in genetically susceptible (C57BL6J) mice. Moreover, the lack of an ongoing epithelial proliferative response suggested that ciliated cell hyperplasia might reflect a selective increase in EGFR-dependent cell survival based on suppression of cell death in this subpopulation of epithelial cells.

### Example 3

### EGFR Signaling and Ciliated Cell Survival in Culture

To determine whether EGFR provides necessary survival signals to ciliated epithelial cells, EGFR blockade was analyzed in tissue culture where macrophage clearance would not obscure detection of apoptotic cells and where signaling events could be better defined. Initial experiments aimed to determine whether EGFR was localized to ciliated epithelial cells in culture as was found in vivo. The epithelial system was reconstituted in vitro using air-liquid interface cultures of airway epithelial cells harvested from mouse trachea. In this system, ciliated β-tubulin positive) cells represented 45±1% of the total cell population, a level that was similar to normal mouse airways (36% for large-sized airways) and to values for mouse tracheal specimens reported previously (Pack et al. (1980) Cell Tissue Res. 208, 65-84). As was the case in vivo, the ciliated epithelial cells in culture exhibited constitutive expression of EGFR and phospho-EGFR along the apical cell membrane and phospho-EGFR was found in this location as well as a nuclear one following activation by ligand (FIG. 5 and data not shown). Others reported that EGFR may also be localized to the basolateral cell membrane in cultured airway epithelial cells (Vermeer et al. (2003) Nature 422, 322-326), but any differences could depend on culture conditions, receptor heterogenicity that influences recognition by different antibodies, or receptor abundance that may influence apical versus basolateral localization (Kuwada et al. (1998) Am. J. Physiol. Cell Physiol. 275, C1419-C1428).

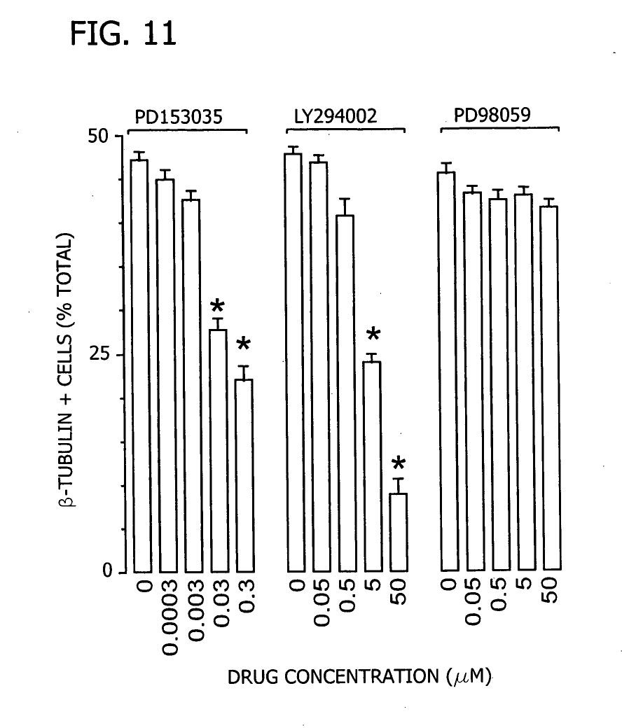

Next defined was the role of EGFR signaling in ciliated epithelial cell growth and survival using treatment with selective inhibitors. Initial experiments to validate inhibitor specificity used cultures that were first removed from complete medium and then stimulated with EGF to maximize EGFR-dependent signals. Under these conditions, it was found that EGFR tyrosine kinase inhibition with PD153035 blocked all downstream signals, while Pk3K inhibition with LY294002 blocked phosphorylation of Akt and MEK1/2 inhibition with PD98059 blocked phosphorylation of ERK1/2 (FIGS. 5 and 6). Then, the effect of these EGFR signals on ciliated cell survival was determined. It was found that treatment with PD153035 caused a dose-dependent loss of ciliated cells out of proportion to the consequent decrease in total epithelial cells (FIGS. 6 and 7). Similar results were obtained with another EGFR-specific inhibitor AG1478 (data not shown). Recognizing that EGFR activation triggers several downstream signaling pathways, mouse tracheal epithelial cell (mTEC) cultures were next treated with inhibitors of Pk3K and MEK1/2 and it was found that only treatment with LY294002 caused a similar loss of ciliated epithelial cells (FIG. 11). Since this culture system does not exhibit significant cell growth at high density, it appears likely that the loss of ciliated epithelial cells was due to decreased cell survival.

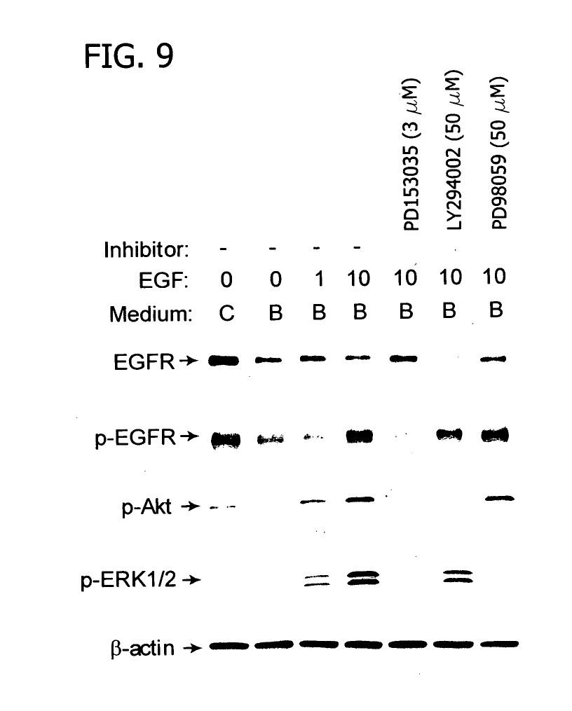

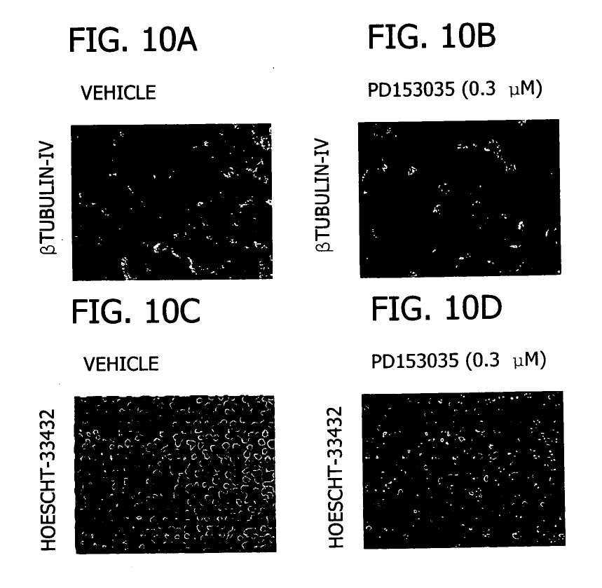

Next tested was whether blockade of EGFR signaling led to concordant changes in the level of apoptosis. In parallel with loss of ciliated epithelial cells, rapid activation of caspase-3 and TUNEL-positive cells (within 6 h) was observed in the same pattern for that of cell loss, i.e., when EGFR or Pk3K but not MEK1/2 signaling was blocked (FIG. 8). In this setting, TUNEL-positive cells were undergoing apoptosis, since this process was blocked by treatment with the caspase inhibitor z-VAD-fmk (FIG. 9) and was associated with caspase-3 and caspase-9 cleavage/activation (FIG. 10) and loss of mitochondrial membrane potential (FIG. 11). These results therefore define an EGFR signaling pathway that protects against ciliated cell apoptosis via selective Pk3K signaling to downstream factors that prohibit mitochondrial dysfunction and consequent programmed cell death. These findings stand in some contrast to reports of EGFR and other receptor signals to ERK1/2 that prevent cell death under other circumstances (Tyner, et al. (2005) Nat. Med. 11:1180-1187; Monick, et al. (2005) J. Biol. Chem. 280:2147-2158; Roux, et al. (2004) Microbiol. Mol. Biol. Rev. 68:320-344).

### Example 4

### EGFR Signaling and Goblet Cell Metaplasia in Vitro and in Vivo

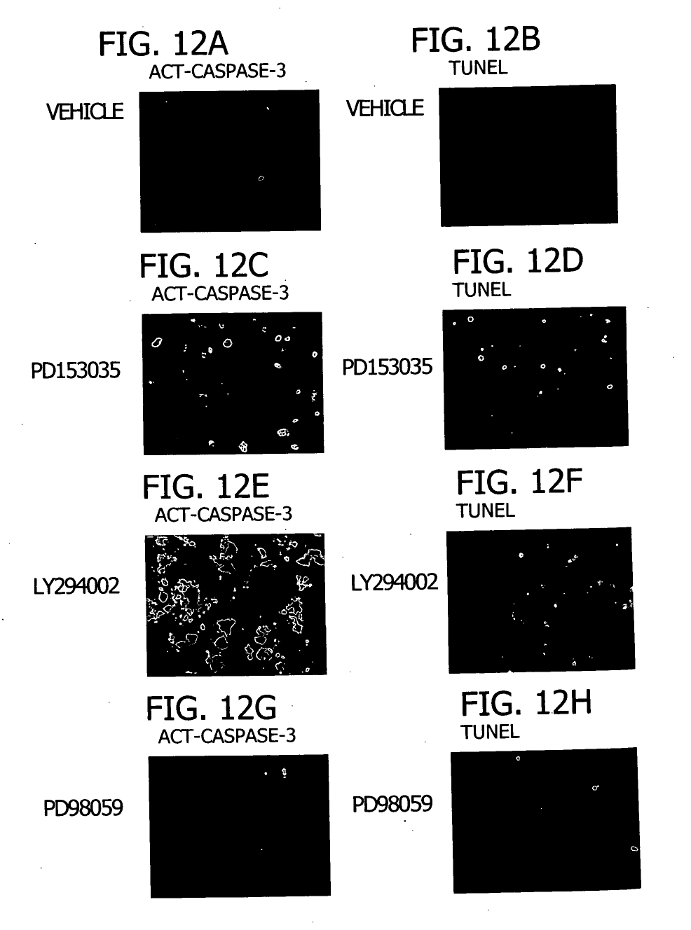

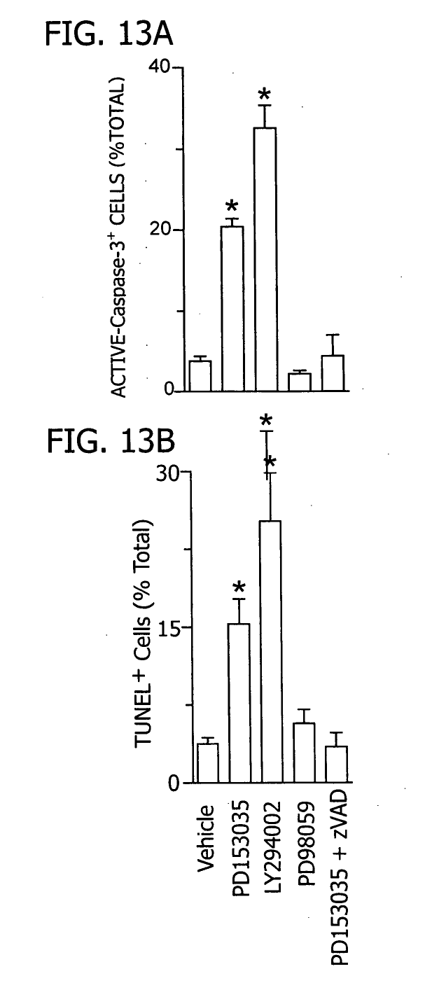

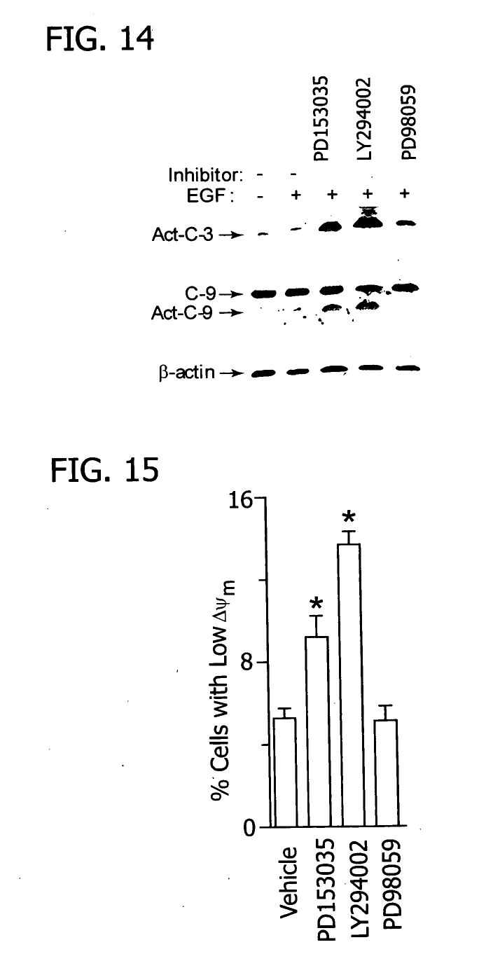

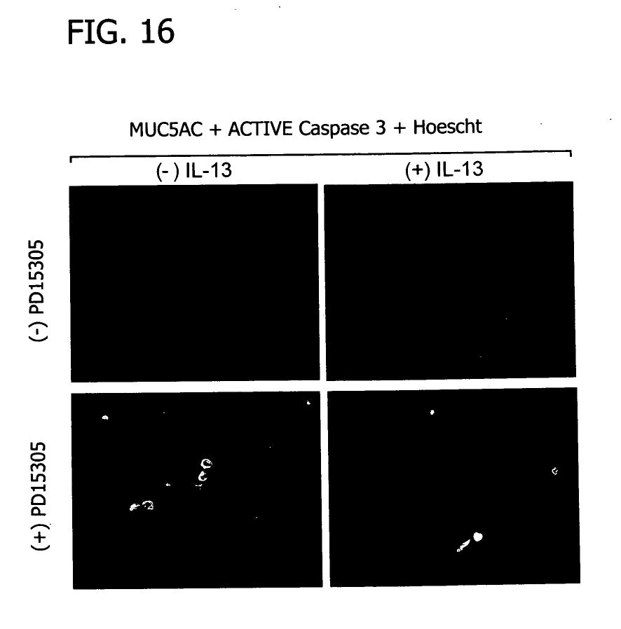

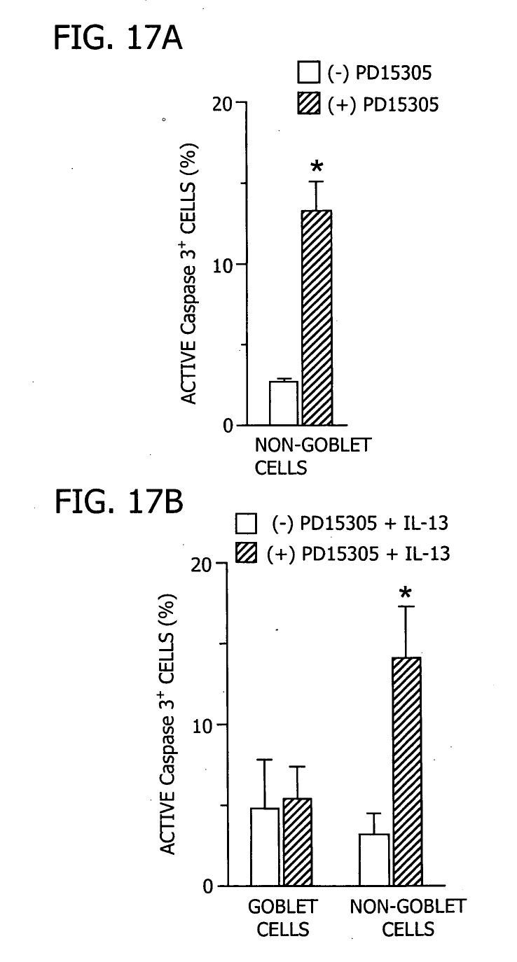

As noted above, the actions of EGFR signaling on ciliated cells did not readily explain the effect of EGFR blockade on goblet cell metaplasia in vivo. Selective EGFR expression and consequent survival function on ciliated cells accounts for inhibition of ciliated cell hyperplasia, but does not account directly for blockade of goblet cell metaplasia. It was next questioned whether EGFR might still have a similar functional effect on goblet cell survival. To test this possibility, advantage was taken of concomitant studies that defined IL-13-dependence of goblet cell metaplasia after viral infection (unpublished observations, E Y Kim and M J Holtzman). This effector pathway therefore overlaps with the one established in studies of mucin production after allergen challenge (Grunig et al. (1998) Science 282, 2261-2263; Wills-Karp et al. (1998) Science 282, 2258-2261). In addition, the capacity of IL-13 treatment to stimulate goblet cell formation in airway epithelial cells cultured from guinea pigs and humans (Laoukili, et al. (2001) J. Clin. Invest. 108:1817-1824; Kondo, et al. (2002) Am. J. Respir. Cell Mol. Biol. 27:536-541; Atherton, et al. (2003) Am. J. Physiol. Lung Cell Mol. Physiol. 285:L730-L739), as well as mice (unpublished observations, J D Morton and M J Holtzman), was recognized. Cultured mouse tracheal epithelial cells were treated with IL-13, and the subsequent development of goblet cells was marked by expression of MUC5AC (Nakanishi et al. (2001) Proc. Natl. Acad. Sci. U.S.A. 98, 5175-51809; Zhou et al. (2001) Am. J. Respir. Cell Mol. Biol. 25, 486-491). Cell death was tracked with activation of caspase 3, since the procedure for the TUNEL reaction appears to decrease mucin content. In contrast to ciliated cells, it was found that goblet cells did not exhibit increased rates of cell death in response to EGFR inhibition (FIG. 12). Quantifying the level of active caspase 3 cells in MUC5AC-positive versus MUC5AC-negative populations indicated that the level of apoptosis was similar in goblet cells with or without EGFR blockade whereas the non-goblet cells (i.e., ciliated epithelial cells) exhibited significant caspase-positive staining under these treatment conditions (FIG. 5C). Moreover, the level of non-goblet cell death was similar with or without IL-13 treatment indicating that the death pathway in ciliated cells is not influenced by IL-13-dependent actions on goblet cell formation.

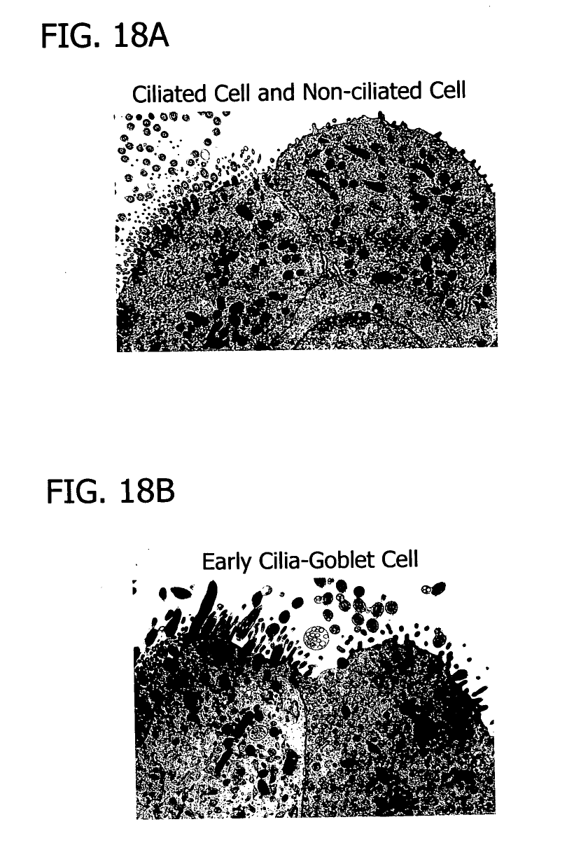

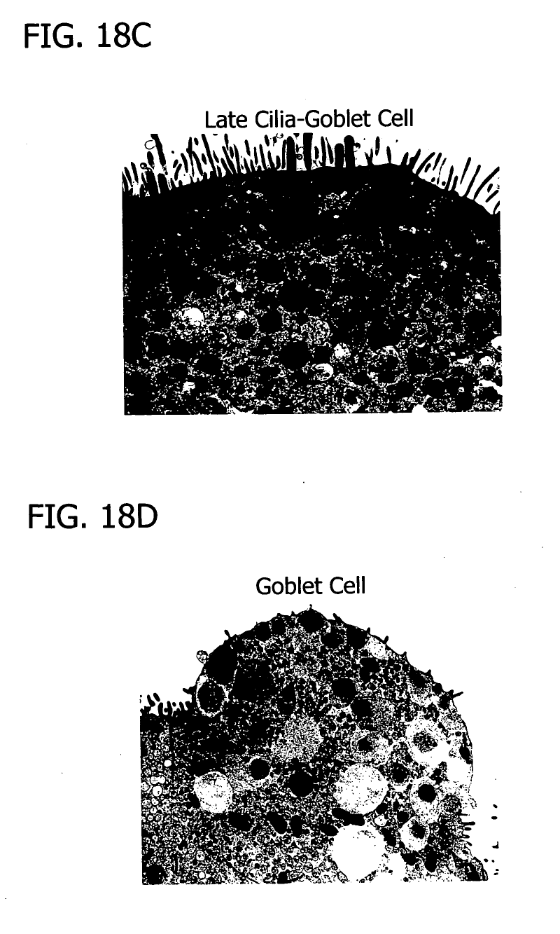

Since EGFR blockade caused no effect on goblet cell survival in vitro, it was reasoned that the potent effects of EGFR blockade on goblet cell metaplasia in vivo might be downstream of EGFR blockade of ciliated cell hyperplasia. Support for that possibility came next when it was found that IL-13 treatment led to the development of cells that transiently shared characteristics of ciliated and goblet cells. Thus, electron microscopy of mTEC cultures provided evidence of a subset of cilia-goblet cells with preservation of cilia and the gradual development of mucous granules under the influence of IL-13 (FIG. 18). These transitional cells were most prominent early (1-2 days) after initiation of IL-13 treatment, while mature goblet cells without cilia were most abundant at later times (5 days) after treatment. The morphologic characteristics of cilia-goblet cells under these conditions appear similar to ciliated cells containing mucous granules found by electron microscopy in airways of allergen-challenged mice (Hayashi, et al. (2004) Virchows Arch. 444:66-73.

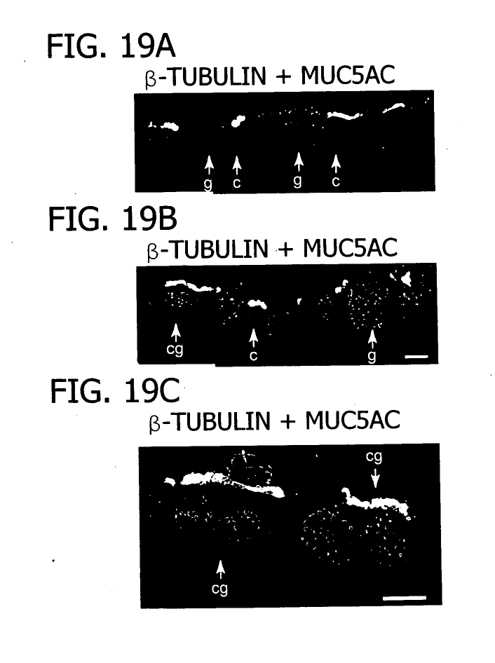

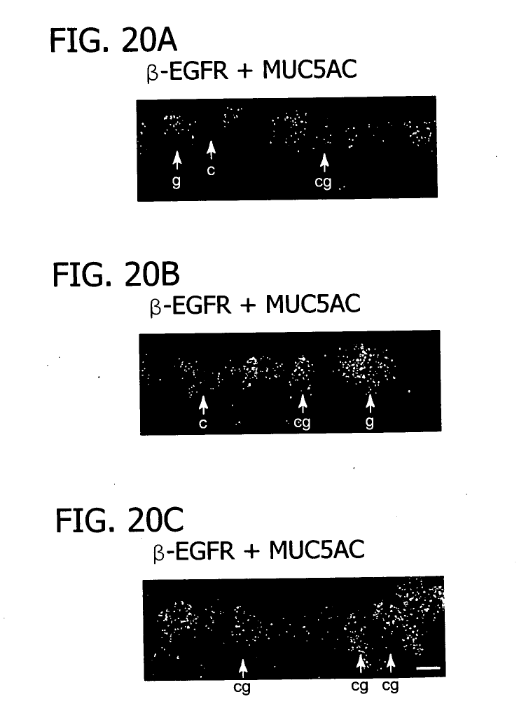

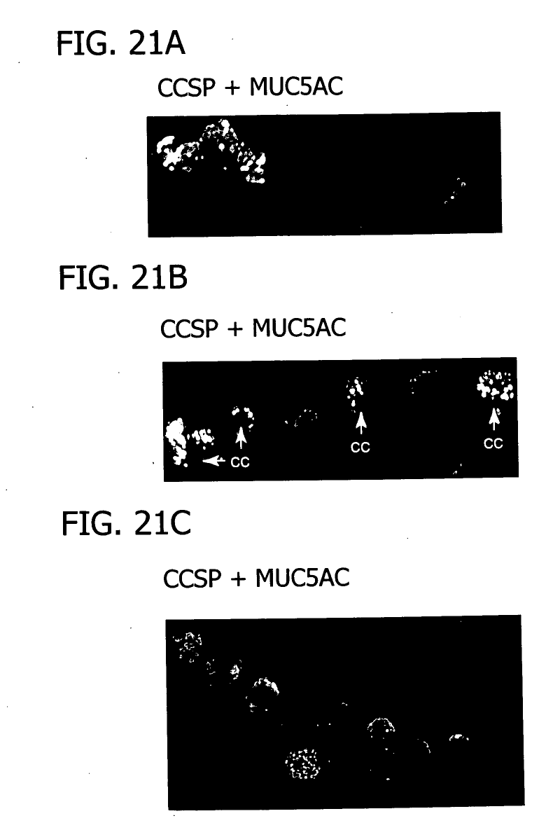

Since airway epithelial cultures were established under conditions that utilize IL-13 treatment to promote goblet cell formation in an environment that would otherwise produce ciliated cells, these cilia-goblet cells were likely being redirected by IL-13 to transdifferentiate from a ciliated to goblet cell phenotype. The possibility that similar transdifferentiation also developed in vivo was confirmed in sections taken from mice exhibiting goblet cell metaplasia after inoculation with SeV. Confocal microscopy indicated that, while the majority of ciliated or goblet cells expressed either β-tubulin or MUC5AC, respectively, there was a subpopulation of epithelial cells that expressed both β-tubulin and MUC5AC (FIG. 19). Similarly, confocal images also indicated that, in general, ciliated but not goblet cells expressed EGFR, but there was an additional subpopulation of cells that expressed both EGFR and MUC5AC (FIG. 20). In each case, multiple confocal sections along the z-axis and 3-dimensional reconstruction were used to confirm co-localization within a single cell. The subpopulation expressing MUC5AC and β-tubulin and/or MUC5AC and EGFR appeared to be in transition, since they did not often reach their characteristic shape and position at the lumenal surface of the mucosal epithelial layer as was found for mature goblet cells. In addition, in these transitioning cells, the mucous granules were often localized in a more basal compartment of the cells versus a more apical location for fully differentiated goblet cells. This morphologic behavior also suggests that these cilia-goblet cells represent goblet cell precursors. As noted previously for allergen-induced goblet cell metaplasia, a subpopulation of epithelial cells with co-expression of CCSP and MUC5AC was also detected (FIG. 21) at levels comparable to detection of cilia-goblet cells (FIG. 23).

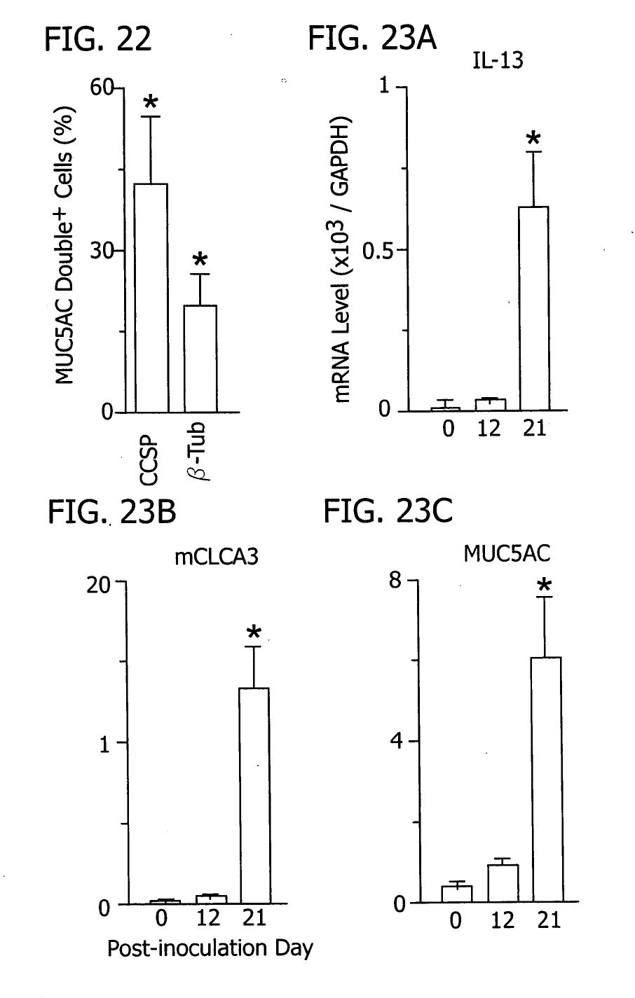

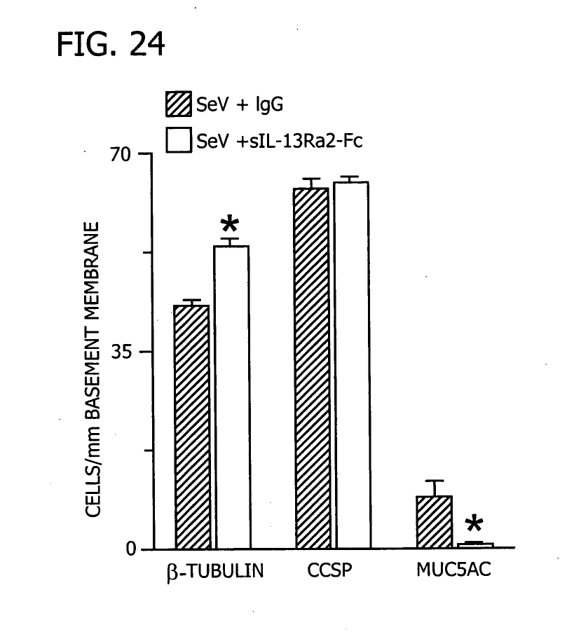

The next aim was to establish whether IL-13 also promotes cilia to goblet cell formation in vivo as it had been observed in vitro. These experiments took advantage of a recombinant soluble IL-13 receptor α2 Fc fusion protein (designated slL-13Rα2-Fc) that acts as a decoy receptor to specifically block IL-13 action when delivered to mice (Grunig et al. (1998) Science 282, 2261-2263; Wills-Karp et al. (1998) Science 282, 2258-2261). Treatment conditions were chosen to be similar to those used for EGFR blockade, so treatment extended from day 12 to 21 after viral inoculation. This time frame also coincides with the induction of IL-13, mCLCA3, and MUC5AC gene expression in concert with the development of goblet cell metaplasia (FIG. 22). Under these conditions, we found that slL-13Rα2-Fc treatment was highly effective in preventing virus-induced goblet cell metaplasia (FIG. 23). However, in some contrast to EGFR blockade, slL-13Rα2 treatment also caused a further increase in the level of ciliated cell hyperplasia (consistent with a block in their movement to goblet cells) and no change in Clara cell levels (consistent with the possibility that goblet cell formation derives at least in part from ciliated cell rather than Clara cell populations). The possibility that other cellular sources (e.g., Clara cells or basal cells) may also contribute to goblet cell metaplasia in this setting cannot be fully excluded, but the close match of ciliated cell increase to goblet cell decrease after IL-13 blockade suggests that transdifferentiation of the ciliated cell population is a significant pathway for goblet cell metaplasia under these conditions. Indeed, together with previous and present evidence of Clara cell expression of mucin genes, the present results may simply provide evidence of additional plasticity of epithelial cell differentiation.

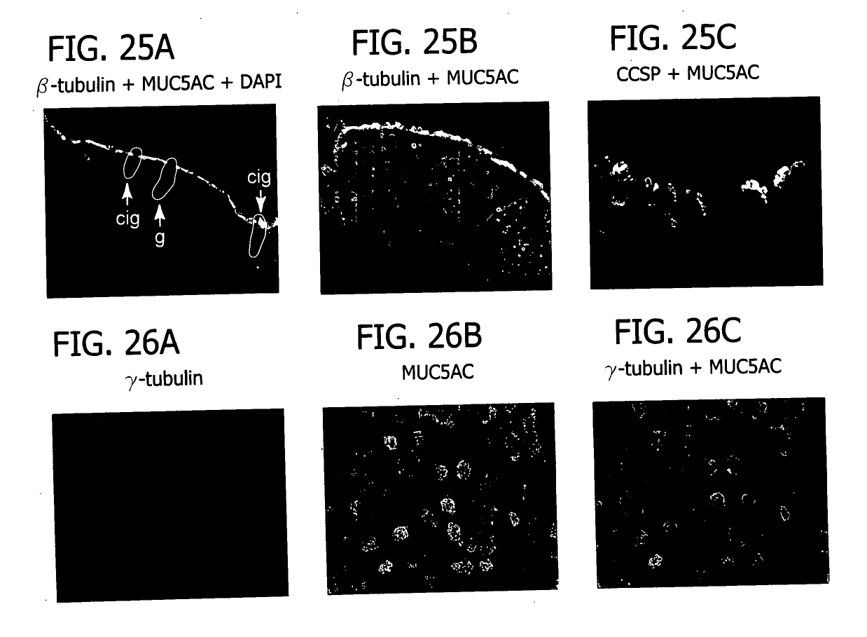

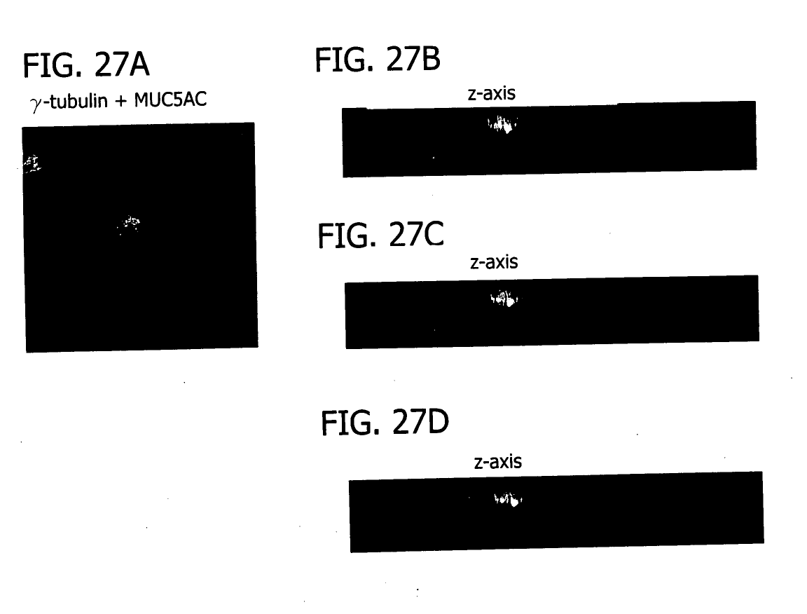

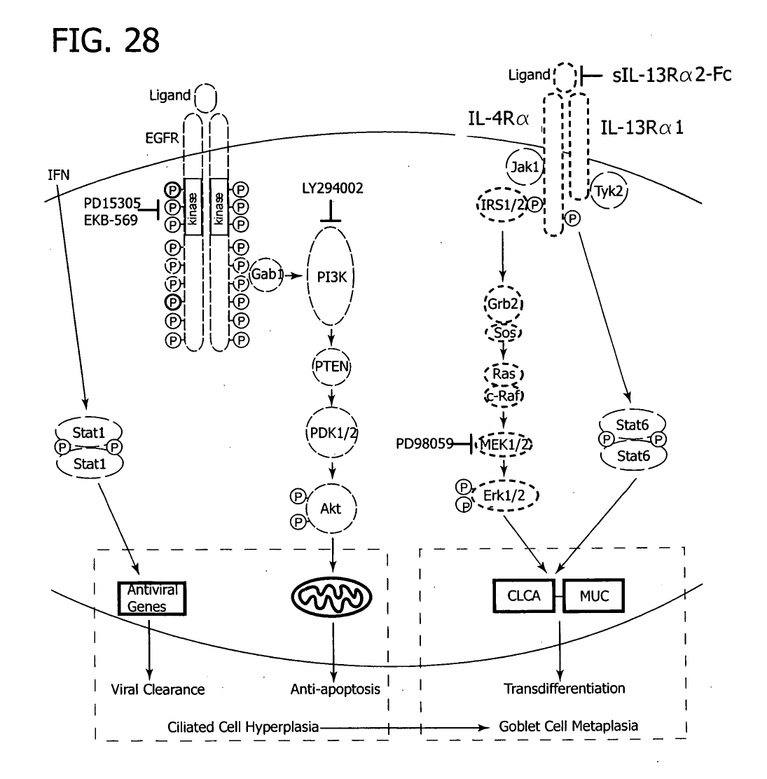

In a final two sets of experiments, the findings were again extended from mice to studies of human subjects. In the first set of experiments, airway tissue from COPD patients that exhibit markedly increased levels of goblet cells in the airway and that provide adequate lung tissue for analysis at the time of lung transplantation were analyzed. By applying the same immunostaining protocol for immunofluorescence and confocal microscopy as for the mouse model, it was found that sections of lung explants from COPD patients also exhibit evidence of β-tubulin-MUC5AC co-expression in a subset of airway epithelial cells (FIG. 25). Luminal staining for β-tubulin in human (or mouse) airways, consistent with the proposal that cilia may be processed by endosomal degradation rather than shedding, was not detected. As noted previously (Boers, et al. (1999) Am. J. Respir. Crit. Care Med. 159), CCSP-MUC5AC co-expression was also found in a subset of epithelial cells. In the second set of experiments, the strategy that was used for mouse studies was again applied and the behavior of human airway epithelial cells in air-liquid interface culture conditions without or with IL-13 was analyzed. In this case, it was found that airway epithelial cells cultured from COPD patients led to the development of a subset of cells that co-expressed β-tubulin and MUC5AC under the influence of IL-13. As noted previously, β-tubulin is localized within the basal bodies of ciliated cells (You, et al. (2004) Am. J. Physiol. Lung Cell. Mol. Physiol. 286:L650-657), and so provides even closer co-localization with MUC5AC found in mucous granules. The same pattern of IL-13-induced co-expression of ciliated and goblet cell markers was found in airway epithelial cells cultured from otherwise healthy lung transplant donors in response to IL-13 even within the first day of IL-13 treatment (FIG. 27). As with the studies of mouse and human tissue, multiple confocal images were examined along the z-axis and 3-dimensional reconstruction to establish co-localization of ciliated and goblet cell markers within a single cell. Thus, similar to the EGFR activation experienced in human subjects, it was found that the downstream mechanism for goblet cell metaplasia found in the mouse model, i.e., IL-13-driven ciliated-to-goblet cell transdifferentiation, appears to have a counterpart in hypersecretory human disease.

What is claimed is:

1. A composition for the treatment of airway hypersecretion comprising an inhibitor of the EGFR signaling pathway, an inhibitor of the IL-13 signaling pathway, and a pharmaceutically acceptable carrier.

2. The composition of claim 1 wherein the inhibitor of the EGFR signaling pathway is an immunopeptide.

3. The composition of claim 1 wherein the inhibitor of the IL-13 signaling pathway is an immunopeptide.

4. The composition of claim 2 wherein the immunopeptide is a polyclonal antibody, a monoclonal antibody, or an antibody fragment.

5. The composition of claim 3 wherein the immunopeptide is a polyclonal antibody, a monoclonal antibody, or an antibody fragment.

6. The composition of claim 2 wherein the anti-EGFR signaling immunopeptide is C225 or EMD55900.

7. The composition of claim 3 wherein the anti-IL-13 signaling immunopeptide is slL-13Rα2-Fc.

8. The composition of claim 1 wherein the inhibitor of the EGFR signaling pathway is an antisense nucleic acid that reduces the expression of EGF or EGFR.

9. The composition of claim 1 wherein the inhibitor of the IL-13 signaling pathway is an antisense nucleic acid that reduces the expression of IL-13, IL-13R, or IL4R.

10. The composition of claim 1 wherein the inhibitor of the EGFR signaling pathway is a tyrosine kinase inhibitor.

11. The composition of claim 10 wherein the tyrosine kinase inhibitor is PD153035, EKB-569, 4-(3-Chloroanilino)-6;7-dimethoxyquinazoline, RG-14620; Tyrphostin 23, Tyrphostin 25, Tyrphostin 46, Tyrphostin 47, or Tyrphostin 51.

12. The composition of claim 1 wherein the inhibitor of the EGFR signaling pathway or the IL-13 signaling pathway is a specific inhibitor of Pk3K.

13. The composition of claim 12 wherein the Pk3K inhibitor is LY294002.

14. The composition of claim 1 wherein the inhibitor of the IL-13 signaling pathway is a PD98059 or LY294002.

15. A process of treating airway hypersecretion in an individual, said process comprising administering an inhibitor of the EGFR signaling pathway and an inhibitor of the IL-13 signaling pathway.

16. The process of claim 15 wherein the inhibitor of the EGFR signaling pathway and the inhibitor of the IL-13 signaling pathway are administered simultaneously.

17. The process of claim 15 wherein inhibitor of the EGFR signaling pathway and the inhibitor of the IL-13 signaling pathway are administered simultaneously as the composition of claim 1.

18. The process of claim 15 wherein at least one inhibitor is an immunopeptide administered in an amount of (i) about 0.05 mg to about 2.5 mg; (ii) about 0.1 mg to about 1 mg; or (iii) about 0.3 mg to about 0.5 mg.

19. The process of claim 15 wherein at least one inhibitor is an antisense inhibitor administered in an amount of (i) about 10 μg/day to about 3 mg/day; (ii) about 30 μg/day to about 300 μg/day; or (iii) about 100 μg/day.

20. The process of claim 15 wherein at least one inhibitor is a tyrosine kinase inhibitor administered in an amount of about 0.1 μg to about 100 mg per kg per administration.

21. The process of claim 15 wherein the inhibitors are administered by injection, inhalation, orally, liposome, or retroviral vector.
